(19) United States
(12) Patent Application Publication
(10) Pub. No.: US 2026/0023074 A1
(43) Pub. Date: Jan. 22, 2026

(54) DOC2B AS A BIOMARKER FOR TYPE 1 AND TYPE 2 DIABETES

(71) Applicant: CITY OF HOPE, Duarte, CA (US)

(72) Inventor: Debbie C. THURMOND, Glendora, CA (US)

(73) Assignee: CITY OF HOPE, Duarte, CA (US)

(21) Appl. No.: 19/250,644

(22) Filed: Jun. 26, 2025

Related U.S. Application Data
(63) Continuation of application No. 17/231,986, filed on Apr. 15, 2021.
Application No. 17/231,986 is a continuation-in-part of application No. 16/968,511, filed on Aug. 7, 2020, now abandoned.
(60) Provisional application No. 62/628,578, filed on Feb. 9, 2018.

(51) Int. Cl. G01N 33/543; A61K 35/39; C12Q 1/6883

(52) U.S. Cl. CPC G01N 33/543; A61K 35/39; C12Q 1/6883; C12Q 2600/158; G01N 2333/4703; G01N 2800/042; G01N 2800/50

(57) ABSTRACT

Disclosed is the use of DOC2B as an early stage biomarker for diagnosing type 1 diabetes (T1D), pre-T1D, type 2 diabetes (T2D), pre-T2D or for assessing the risk of T1D, pre-T1D, T2D, or pre-T2D. Also disclosed are methods of in vivo diagnosing T1D, pre-T1D, T2D, or pre-T2D or assessing the risk of T1D, pre-T1D, T2D, or pre-T2D by detecting a reduced level of DOC2B expression in a biological sample including blood, plasm, serum, platelets, and pancreatic islets.

### PRIORITY CLAIM

This application is a continuation of U.S. patent application Ser. No. 17/231,986, filed Apr. 15, 2021, which is a continuation-in-part of U.S. patent application Ser. No. 16/968,511, filed on Aug. 7, 2020, which is a national phase entry of PCT Application No. PCT/US2019/017364, filed on Feb. 8, 2019, which claims priority to U.S. Provisional Application No. 62/628,578, filed on Feb. 9, 2018, the contents of which are incorporated by reference herein in their entireties, including drawings.

### SEQUENCE LISTING

This application contains a ST.26 compliant Sequence Listing, which was submitted in XML format via Patent Center, and is hereby incorporated by reference in its entirety. The XML copy, created on Aug. 13, 2025, is named Substitute SL 0544358181 US03.xml and is 24,000 bytes in size.

### FIELD OF THE INVENTION

The present invention relates to early detection, prevention or delaying the onset, and treatment of diabetes including type 1 diabetes (T1D) or pre-T1D and type-2 diabetes (T2D) or pre-T2D.

### BACKGROUND

T1D is characterized by autoimmune destruction of β-cell mass, and the preclinical phase of T1D is marked by declining β-cell function [1,2]. Studies of early interventional in T1D have shown limited effectiveness, yet have generally shown greater success in subjects that retain greater insulin secretory capacity, and in those with the shortest time since clinical onset of disease [3,4]. However, prevention efforts to protect β-cell mass are hindered by the limited availability of early biomarkers to accurately predict β-cell destruction and subsequent progression to clinical disease. Currently, the clinical auxiliary diagnostic markers for T1D are family history, Human Leukocyte Antigen (HLA) genetic screening, and serum autoantibodies against β-cells antigens, including insulin, glutamic acid decarboxylase (GAD), tyrosine phosphatase-like insulinoma antigen 2 (IA-2), and zinc transporter 8 (ZnT8). Asymptomatic individuals with seroconversion to any two autoantibodies have >80% of the risk for the clinical onset of T1D. Nevertheless, autoantibodies in predicting T1D risk have several limitations: 1) high heterogeneity in T1D progression, 2) the inability to track the levels of autoantibodies after seroconversion and 3) limited screening availability for the first-degree relatives of T1D. Meanwhile, type 2 diabetes (T2D) has reached the stage of a global pandemic. Approximately 10% of the total US population (about 30 million people) are impacted by T2D and 86 million more are in the stage of prediabetes. Therefore, there is an unmet clinical need in detecting T1D and T2D at an early stage, preventing or delaying the onset of T1D and T2D, and treating T1D and T2D. The disclosed technology can be applied to T1D or T2D diagnosis, prognosis and treatment.

### SUMMARY OF THE INVENTION

In one aspect, disclosed herein is a method of diagnosing T1D, pre-T1D, T2D, or pre-T2D at an early stage in a subject or assessing the risk of T1D, pre-T1D, T2D, or pre-T2D in a subject. The method entails the steps of detecting the level of DOC2B expression in a biological sample collected from the subject, and comparing the level of DOC2B expression with that of a healthy, control subject or with a pre-set threshold level, wherein a reduced level of DOC2B expression indicates that the subject is suffering from or at an elevated risk of suffering from T1D, pre-T1D, T2D, or pre-T2D. In some embodiments, the biological sample includes blood, plasma, serum, platelets, and pancreatic islets. In some embodiments, detecting the level of DOC2B expression comprises detecting the level of DOC2B protein or the level of DOC2B mRNA in the biological sample. In some embodiments, the DOC2B protein level in the biological sample is determined by a high-throughput screening ELISA using one or more antibodies disclosed herein. In some embodiments, the level of DOC2B expression is reduced by at least 20%, at least 30%, at least 40%, at least 50%, at least 60%, at least 70%, at least 80%, at least 90%, or at least 95%.

In a related aspect, disclosed herein is a method of treating T1D, pre-T1D, T2D, or pre-T2D or delaying the onset of T1D, pre-T1D, T2D, or pre-T2D in a subject. The method entails the steps of detecting the level of DOC2B expression in a biological sample collected from the subject, comparing the level of DOC2B expression with that of a healthy, control subject or with a pre-set threshold level, wherein a reduced level of DOC2B expression indicates that the subject is suffering from or at an elevated risk of suffering from T1D, pre-T1D, T2D, or pre-T2D and administering one or more T1D or T2D treatments to the subject who is determined to suffer from T1D, pre-T1D, T2D, or pre-T2D or at an elevated risk of T1D, pre-T1D, T2D, or pre-T2D. In some embodiments, the biological sample includes blood, plasma, serum, platelets, and pancreatic islets. In some embodiments, detecting the level of DOC2B expression comprises detecting the level of DOC2B protein or the level of DOC2B mRNA in the biological sample. In some embodiments, the DOC2B protein level in the biological sample is determined by a high-throughput screening ELISA using one or more antibodies disclosed herein. In some embodiments, the level of DOC2B expression is reduced by at least 20%, at least 30%, at least 40%, at least 50%, at least 60%, at least 70%, at least 80%, at least 90%, or at least 95%. In some embodiments, the one or more treatments include transplanting healthy, functional β-cells or pancreatic islets to the subject.

In another aspect, disclosed herein is a method of assessing early stage pancreatic β-cell destruction or loss of functional β-cells in a subject. The method entails the steps of detecting the level of DOC2B expression in a biological sample collected from the subject, and comparing the level of DOC2B expression with that of a healthy, control subject or with a pre-set threshold level, wherein a reduced level of DOC2B expression indicates pancreatic β-cell destruction or loss of functional β-cells in the subject. In some embodiments, the biological sample includes blood, plasma, serum, platelets, and pancreatic islets. In some embodiments, detecting the level of DOC2B expression comprises detecting the level of DOC2B protein or the level of DOC2B mRNA in the biological sample. In some embodiments, the DOC2B protein level in the biological sample is determined by a high-throughput screening ELISA using one or more antibodies disclosed herein. In some embodiments, the level of DOC2B expression is reduced by at least 20%, at least 30%, at least 40%, at least 50%, at least 60%, at least 70%, at least 80%, at least 90%, or at least 95%.

In yet another related aspect, disclosed herein is an ELISA kit for detecting the DOC2B level in a biological sample obtained from a subject. The ELISA kit includes one or more antibodies disclosed herein. In some embodiments, the ELISA kit further includes reagents and/or secondary antibodies for performing the ELISA. In some embodiments, the ELISA kit further includes instructions for using the kit. In some embodiments, the biological sample includes blood, plasma, serum, platelets, and pancreatic islets. In some embodiments, the subject is at an elevated risk of T1D, pre-T1D, T2D, or pre-T2D or suffers from T1D, pre-T1D, T2D, or pre-T2D.

### BRIEF DESCRIPTION OF THE DRAWINGS

FIGS. 1A-1B show that DOC2B protein abundance was reduced in platelets of pre-diabetic NOD mice. Platelets were isolated from 16-week (FIG. 1A) or 13-week (FIG. 1B) old group-housed female NOD and age-matched NOR mice and proteins were resolved on SDS-PAGE for immunoblotting. DOC2B levels were quantified relative to tubulin immunoblotting in the same lane. Dashed vertical lines indicate splicing of lanes from within the same gel exposure. Data are shown as means±SEM (n=3-6 mice per group); *p<0.05.

FIGS. 2A-2C show that islets from young pre-diabetic NOD mice were deficient in DOC2B protein. Islets were isolated from 16-week (FIG. 2A), 13-week (FIG. 2B) or 7-week (FIG. 2C) old group-housed female NOD and age-matched NOR mice and proteins were resolved on SDS-PAGE for immunoblotting. DOC2B levels were quantified relative to tubulin loading in the same lane. Dashed vertical lines indicate splicing of lanes from within the same gel exposure. Data are shown as means±SEM for DOC2B (n=3-7 mice per group); *p<0.05.

FIG. 3 shows that DOC2B protein abundance was reduced in platelets from new-onset pediatric T1D human subjects. Platelets were isolated from new-onset T1D patients at the time of diagnosis (“Diagnosis”) and 7-10 weeks later (“First Follow-up”), and from matched controls (“Control”). Platelet proteins were resolved on SDS-PAGE for immunoblotting. Standard curves were generated using recombinantly-expressed and purified human DOC2B protein on each gel to confirm that the band intensities of DOC2B in human platelets fell within the dynamic range of the curve on the same gel. DOC2B was quantified relative to protein loading determined by Ponceau S staining in the same lane (37-68 kDa segment). Dashed vertical lines indicate splicing of lanes from within the same gel exposure. Data are shown as means±SEM for DOC2B (n=11-14 per group (gender-combined group, 8 males per group, 3-6 females per group); *p<0.05, Diagnosis vs. Control; #p<0.05 Follow-up vs. Control).

FIGS. 4A-4B show that DOC2B protein and mRNA abundance was reduced in adult human islets subjected to treatment with pro-inflammatory cytokines. Human adult cadaveric islets were incubated under control conditions or with pro-inflammatory cytokines for 72 h at 37° C. Islet protein lysates were resolved by SDS-PAGE for immunoblotting (FIG. 4A) or for RNA extraction and qRT-PCR analysis (FIG. 4B). In addition to hDOC2B and tubulin, iNOS levels were also evaluated by immunoblotting. Bars represent mean±SEM for 4 or 5 independent sets of human islets evaluated for protein and mRNA analyses, respectively; ****p<0.0001, **p<0.002.

FIGS. 5A-5D show that DOC2B protein levels were reduced in islets from pediatric T1D humans. Slides obtained from nPOD comprised of early-onset T1D and age-matched non-diabetic human pancreata were immunostained for the presence of DOC2B, insulin or glucagon positive cells. FIG. 5A shows representative images, low power images scale bar=100 μm, higher magnification images scale bar=25 μm. FIG. 5B shows tabulated relative intensities; n=3 donors, *p<0.05. FIG. 5C shows the number of DOC2B-positive β-cells p=not significant, (N.S.). FIG. 5D shows DOC2B reduction in pre-type 2 diabetes.

FIGS. 6A-6D show that DOC2B levels in adult T1D human platelets and plasma were increased after clinical islet transplantation. Platelets obtained from two clinical islet transplant recipients prior to (Day 0) islet infusion, or on Day 30 and Day 75 post-infusion, were evaluated by quantitative immunoblotting for DOC2B protein content: subject COH-027 (FIGS. 6A, 6C), and subject COH-028 (FIGS. 6B, 6D). Ponceau S staining and GAPDH show the relative protein loading of the membranes used for immunoblotting. The data show the same trends, meaning that either platelets or plasma provided the same predictive information.

FIG. 7 shows that platelet proteins from children with T1D and age/gender/BMI matched controls were isolated at Diagnosis and First Follow-up 7-10 weeks later, then resolved on SDS-PAGE for immunoblotting for STX4. Standard curves were included using recombinantly-expressed and purified human STX protein on each gel with band intensities of STX4 in human platelets falling within the dynamic range of the curve on the same gel. Dashed vertical lines indicate splicing of lanes from within the same gel exposure. Data are shown as means±SEM. n=10-13 per gender-combined group, 5-7 males per group, 3-6 females per group); *p<0.05, Diagnosis vs. Control; #p<0.05 Follow-up vs. Control.

FIG. 8 shows a diagram of the epitopes on human DOC2B.

FIG. 9 shows the alignment of DOC2B and DOC2A amino acid sequences (SEQ ID NOS: 1-7).

FIG. 10 shows the alignment of DOC2B amino acid sequences across species (SEQ ID NOS: 1-3 and 8).

FIG. 11 shows immunofluorescent detection of DOC2B in mouse p cells and mouse pancreas.

FIG. 12 shows immunoblot detection of DOC2B with Antibody #2.

FIG. 13 shows immunoblot detection of DOC2B with Antibody #2 rabbit 12727 and rabbit 12728.

FIG. 14 shows that commercially available human DOC2B ELISAs are not suitable for detecting DOC2B.

FIG. 15 shows validation of custom DOC2B Antibodies #1, #2, #3, and #4 in a denatured form. All custom rabbit polyclonal DOC2B antibodies detected a single DOC2B protein band of the appropriate molecular weight under denaturing condition. *BP: blocking peptide.

FIG. 16 shows that custom rabbit anti-human DOC2B antibodies recognized the native form of human recombinant DOC2B.

FIG. 17 shows that sandwich ELISA was developed using custom rabbit polyclonal anti-human DOC2B antibodies.

FIG. 18 shows that extracellular vesicles from human islets contained DOC2B.

FIG. 19 shows DOC2B-laden EVs were generated from cultured insulin-producing β-cells.

### DETAILED DESCRIPTION

The following description of the invention is merely intended to illustrate various embodiments of the invention. As such, the specific modifications discussed are not to be construed as limitations on the scope of the invention. It will be apparent to one skilled in the art that various equivalents, changes, and modifications may be made without departing from the scope of the invention, and it is understood that such equivalent embodiments are to be included herein.

The term “subject” or “patient” as used herein can be any individual mammal, including but not limited to human, canine, rodent, primate, swine, equine, sheep, and feline. In a particular embodiment, the subject is human.

The terms “treat,” “treating,” and “treatment” as used herein with regard to a condition refer to preventing the onset of the condition, alleviating the condition partially or entirely, or eliminating, reducing, or slowing the development of one or more symptoms associated with the condition.

Double C2 domain protein-β (DOC2B) has DOC2A and DOC2B isoforms. DOC2B is ubiquitously expressed soluble protein. C2 domains of DOC2B bind to phospholipids, Ca²⁺, and SNAREs and serves as an exocytosis regulator. DOC2B is known to regulate islet function. DOC2B deficiencies in pancreatic β-cells show defects in insulin secretion and regulation of glucose homeostasis. DOC2B enrichment in pancreatic β-cells shows enhancement in both phases of insulin secretion.

As disclosed herein, DOC2B levels are deficient in platelets of new-onset pediatric T1D patients. DOC2B levels are reduced in T1D- and pro-inflammatory cytokine-stressed human islet β-cells. DOC2B abundance has already decreased in both islet and platelets of prediabetic mice. DOC2B deficiency in established adult T1D human platelets is increased after clinical islet transplantation. DOC2B abundance is reduced in both human platelets and plasma of pre-T2D. DOC2B levels are reduced in T2D human islet. DOC2B is capable of reporting defective β-cell mass.

C-peptide, the biomarker used as the standard of care for evaluation of β-cell dysfunction, is insufficient to detect early pre-type 2 diabetes. The DOC2B abundance is closely related to β-cell function. There is a significant correlation between the decline in DOC2B abundance in blood-derived platelets and plasma and the loss of β-cell function in early-onset human pre-type 2 diabetic subjects. DOC2B deficiency is detectable in very early pre-diabetic NOD mice (mouse model of type 1 diabetes). The mechanism by which DOC2B is released into the circulation involves secretion of DOC2B-laden EVs from β-cells. Accordingly, DOC2B can serve as a biomarker for early β-cell dysfunction.

As disclosed herein, attenuated DOC2B in the circulation may serve as a diagnostic or prognostic biomarker in preclinical or clinical type 2 diabetes as well as in preclinical or clinical type 1 diabetes and in cancer patients with otherwise undetectable “weak” β-cells. In some embodiments, the cancer patient is suitable for treatment using checkpoint inhibitor therapies. A severe, often lethal form of diabetes occurs rapidly in at least 2% of patients with no prior history of diabetes and there is no screen for determining who will be afflicted. Moreover, patients with existing pre-type 2 diabetes characterized by an intermediate level of fasting hyperglycemia between 100-125 mg/dl blood glucose and frank type 2 diabetes with >126 mg/dl blood glucose are severely adversely afflicted by checkpoint inhibitors, with their diseases escalating in severity. These severe effects of the checkpoint inhibitors are permanent alterations. Accordingly, the technology disclosed herein can be applied for diagnosis of dysfunction of β-cells/diabetes susceptibility in normoglycemic patients under surveillance for diabetes, as well as for disease monitoring in those with diabetes/pre-diabetes or cancer, including patients with dysglycemia, type 1 or type 2 diabetes, cancer patients treated with immune-checkpoint inhibitors or agents that lead to diabetes.

Given the unavailability of platelets in repositories, human plasma was screened and showed clear detection of DOC2B in human plasma samples. It was further determined that DOC2B deficiency is detectable in very young (7-week-old) pre-diabetic NOD mice (a model of T1D); while C-peptide was unable to detect any changes in β-cell function at this very early stage of the disease onset. These data indicate that reduction of DOC2B precedes C-peptide decline in β-cells. Furthermore, severe DOC2B deficit was previously demonstrated in blood-derived platelets at diagnosis and restoration upon islet transplantation in T1D patient recipients. This was recapitulated in the plasma collected from the same T1D patient recipients and timepoints. DOC2B levels were observed to be restored within 30-days post transplantation. To determine the mechanism by which DOC2B is transported from the β-cells into the plasma, extracellular vesicles (EVs) from conditioned medium of human non-diabetic and pre-diabetic pancreatic islets for DOC2B protein content were analyzed. Indeed, DOC2B levels were lower in the prediabetic islet-derived EVs when compared to the non-diabetic islet-derived EVs. DOC2B-laden EVs from liver, β-cells, brain and skeletal muscle were also assessed; and DOC2B content in EVs released by these other cell types was nearly undetectable. These data indicate circulating DOC2B in plasma-derived EVs is predominantly of β-cell-source. Thus, DOC2B is a novel biomarker for a new diagnostic that could replace existing standard of care for β-cell function (C-peptide) and have broad utility for metabolic aberration detection earlier in disease progression so as to permit earlier intervention and prevention of conversion to clinical diabetes as well as checkpoint inhibitor-induced diabetes.

Disclosed herein is a correlation between functional β-cell mass and the level of DOC2B expression in a biological sample, where the reduction of DOC2B expression indicates the loss of functional β-cell mass, thereby leading to the early diagnosis of T1D, pre-T1D, T2D, or pre-T2D. The DOC2B expression level is reduced even prior to the onset of T1D, pre-T1D, T2D, or pre-T2D. Therefore, DOC2B can be used as an early biomarker not only to report the status of T1D, pre-T1D, T2D, pre-T2D but also to prevent or delay the onset of T1D, pre-T1D, T2D, or pre-T2D. Additionally, the DOC2B expression level in blood, plasma, serum and/or platelets closely correlates with the DOC2B expression in pancreatic islets. Therefore, the method disclosed herein allows a non-invasive, early diagnosis of T1D, pre-T1D, T2D, or pre-T2D or early assessment of T1D, pre-T1D, T2D, or pre-T2D risk from a blood, plasma, serum or platelet sample.

In healthy β-cells, insulin secretion requires soluble N-ethylmaleimide-sensitive factor-attachment protein receptor (SNARE) proteins and associated accessory regulatory proteins to promote the docking, priming, and fusion of insulin vesicles at the plasma membrane. Two target membrane (t)-SNARE proteins, Syntaxin1/4 and SNAP25/23, and one vesicle associated (v-SNARE) protein, VAMP2, constitute the SNARE core complex [5]. Assembly of the SNARE complex occurs when one v-SNARE binds two cognate t-SNARE proteins in a heterotrimeric ratio [6]. SNARE complex assembly is also facilitated by Double C2-domain protein p (DOC2B) [7,8]. It has been established that in animal models, deficiencies in DOC2B result in glucose intolerance and insulin secretion defects [9,10]. Conversely, overexpression of DOC2B using global transgenic mouse models enhances insulin secretion and peripheral glucose uptake [11]. Although DOC2B deficiency in rodents has been linked to T2D [12], the association between DOC2B protein levels and T1D is still unknown.

Deficient first-phase insulin secretion is a hallmark of preclinical T1D [1,2], thus, the ability to assess early pancreatic β-cell destruction is critically important for predicting disease onset. Currently, risk prediction for T1D relies heavily on family history, genetic screening, and the presence of antibodies against β-cell antigens that often appear relatively late in the progression of disease. The use of autoantibodies in evaluating T1D risk is limited, as >50% of autoantibody-positive patients remain disease-free, even at 5 years follow up [13]. Risk scores have been established [14], but remain insufficient to provide an accurate prognosis, nor an accurate measurement of β-cell health, as many autoantibody-positive individuals are slow to progress through the stages [15] of preclinical disease. To improve early prediction of T1D, ongoing studies seek to investigate the levels of circulating factors that reflect declining β-cell health, such as proinsulin [16], HSP-90 [17], and unmethylated insulin DNA [18] as potential biomarkers of T1D.

Another potential source of biomarkers is the blood-derived plasma or platelet, which is currently being investigated in diseases such as Alzheimer's disease [19] and cancer [20], and has been implicated in T1D. Changes in the platelet proteome and morphology have been noted in T1D; for instance, altered intracellular Ca²⁺ [21], enhanced formation of microparticles [22], and altered morphology [23] have been reported to result in platelet hyper-reactivity and development of vasculopathies. Importantly, platelets harbor many of the same exocytosis proteins as the pancreatic β-cell, including SNARE isoforms and regulatory accessory proteins [24].

The ability to detect β-cell destruction is critical in accurately predicting prognosis during the preclinical phase of T1D and T2D, hence the current need for additional early biomarkers. As described herein, DOC2B protein levels are substantially reduced in plasma, platelets and islets from pre-diabetic NOD mice vs. NOR control mice. Furthermore, it is shown that levels of human DOC2B are significantly lower at the time of diagnosis in plasma or platelets of new-onset T1D and pediatric patients than platelets from matched control subjects. Notably, DOC2B levels are reduced at 7-10 weeks post-diagnosis, despite therapeutic remediation of hyperglycemia in the human subjects. Consistent with this, islet DOC2B protein levels are reduced in pancreatic tissue samples from T1D patients compared to matched controls. Loss of DOC2B protein and mRNA can be recapitulated by exposure of non-diabetic human islets to pro-inflammatory cytokines ex vivo, suggesting that the inflammatory milieu in pre-diabetic and T1D humans may cause DOC2B loss. Remarkably, clinical islet transplant recipients exhibit a restoration of DOC2B levels in platelets, compared with their own nearly undetectable levels of platelet DOC2B prior to receiving the transplanted islets. These data suggest that DOC2B protein can be a biomarker of pre-diabetes and T1D, with the levels possibly reporting relative functional β-cell mass.

Thus, biomarkers of β-cell destruction in blood have more clinical potential than those in pancreatic islets, as islet procurement is not feasible for routine diagnosis; therefore, the correlation between DOC2B protein abundance in blood-derived platelets and pancreatic islets of T1D mice and humans is investigated. As shown in the working examples, protein abundance of DOC2B is reduced in plasma, platelets and islets from humans with new-onset T1D, compared to matched controls. DOC2B levels are substantially increased in T1D human platelets after transplantation, when C-peptide levels are markedly increased. These data provide a correlation between restored levels of DOC2B in patients having received successful clinical islet transplants, with C-peptide confirming that the islet beta cells transplanted were functioning well.

As disclosed herein, an association between T1D or pre-T1D and levels of an exocytosis protein in blood-derived plasma, platelets and pancreatic islets is established. Reduced DOC2B in islets is indicative of deficient islet functional health [9]. Strikingly, plasma or platelet DOC2B levels in islet transplant recipients correlated with the presence of a functional islet mass. This correlative finding supports the possibility that the plasma or platelet DOC2B stems not necessarily from the pancreas per se, since islets are grafted into the liver in these human recipients, but that the plasma or platelets and/or precursor megakaryocytes may be sampling DOC2B from the islets irrespective of islet location. It also remains possible that the increased DOC2B content stems from “rested” native residual islets of the transplanted patients. However, this is inconsistent with the pediatric platelet data showing that even after insulin therapy to ameliorate new-onset hyperglycemia, DOC2B levels remained deficient. Mechanistically, questions arise as to how plasma, platelets and islets “communicate” to determine DOC2B levels. Supporting the concept of platelet-islet communication, it has been demonstrated that islet transplantation in T1D patients stabilizes platelet abnormalities, as transplant recipient platelets show normal volume and activation [33]. Indeed, β-cells release exosomes as a way of shuttling various miRNAs, mRNAs, and proteins to targeted peripheral cells [34]. R-cell exosomes were also recently shown to carry proteins such as GAD-65, IA-2, and proinsulin, to dendritic cells, which then become activated [35]. Furthermore, platelets can selectively absorb proteins from the blood [36]. In fact, platelet sequestration of tumor-specific proteins was detected in animals harboring small tumors [36]. Notably, a direct interaction between platelets and pancreatic β-cells has been reported, and protein from platelets was shown to be transferred to β-cells [37].

The concept of DOC2B as a biomarker is novel because DOC2B levels in plasma, platelets and islets are significantly decreased in normoglycemic NOD mice months before their conversion to T1D. Female NOD mice typically convert to T1D between 18-24 weeks of age, but as early as 5 weeks of age, NOD mouse islets show signs of insulitis, resulting from an initial phase of pancreatic inflammation that reduces β-cell function and mass [38]. C-peptide is unable to detect this insulitis however, whereas DOC2B does serve as a readout at this very early stage of disease. Given that DOC2B content in human islets decreased upon islet exposure to pro-inflammatory cytokines, which was sufficient to evoke iNOS expression, it is possible that the cytokine-induced drop in islet DOC2B signals reduced islet viability. Although it has been demonstrated by multiple groups that whole-body DOC2B knockout mice show deficient glucose-stimulated insulin secretion [9,10], β-cell mass was not evaluated. While it is also possible that DOC2B expression is genetically repressed in NOD mice, the genetics of NOD mice have been well studied and DOC2B was not identified as deviating from control [39]. DOC2B mRNA expression was also decreased in response to pro-inflammatory cytokine exposure in non-diabetic human islets, suggesting that DOC2B might undergo transcriptional repression during T1D development.

DOC2B protein level in a biological sample can be detected by a high-throughput screening ELISA using the antibodies disclosed herein. The ELISA has an improved accuracy and reliability due to the use of antibodies having less cross-reactivity and fewer non-specific bindings such that the assay has little or no background noise for the detection of DOC2B protein level in the sample. The ELISA results are validated by quantitative immunoblotting of known plasma samples.

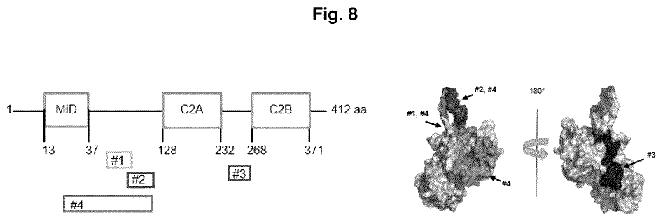

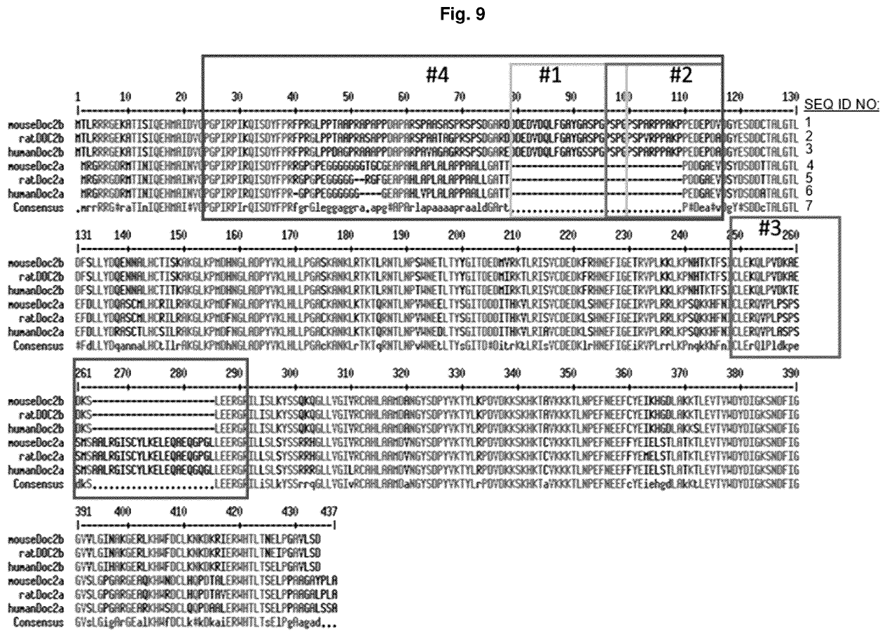

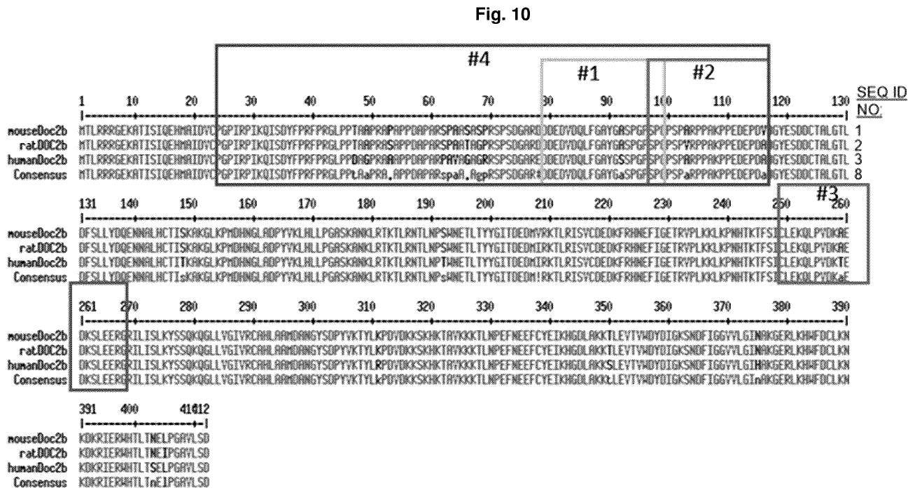

FIG. 8 illustrates the design of the antibodies used in the ELISA. Computer programs for modeling the tertiary structure of DOC2B, including alignment of C2AB containing proteins by Cluster W: information from Vaidehi's core (Supriyo) was used. The 4 antibodies disclosed herein bind to the following antigens: Antibody #1 binds to human DOC2B amino acid sequence AA 79-99, Antibody #2 binds to human DOC2B amino acid sequence AA 96-116, Antibody #3 binds to human DOC2B amino acid sequence AA 249-267 for detection of C2AB, and Antibody #4 binds to human DOC2B amino acid sequence AA 23-62, 55-92, and 82-116. The corresponding epitope sequences are as follows:

#1 hDOC2B(79-99):
(SEQ ID NO: 9)
DDEDVDQLFGAYGSSPGPSPG-Cys (22 aa)
#2 hDOC2B(96-116):
(SEQ ID NO: 10)
Cys-PSPGPSPARPPAKPPEDEPDA (22 aa)
#3 hDOC2B(249-267):
(SEQ ID NO: 11)
CLEKQLPVDKTEDKSLEER (19 aa)
#4 hDOC2B(23-116):
(23-62):
(SEQ ID NO: 12)
CPGPIRPIKQISDYFPRFPRGLPPDAGPRAAAPPDAPARP (40 aa),
(55-92):
(SEQ ID NO: 13)
PPDAPARPAVAGAGRRSPSDGAREDDEDVDQLFGAYGS-Cys (39 aa),
and
(82-116):
(SEQ ID NO: 14)
Cys-DVDQLFGAYGSSPGPSPGPSPARPPAKPPEDEPDA (36 aa).

Accumulating evidence suggests that intrinsic β-cell stresses, secretory dysfunction, and inflammation are the early players during the progression of the T2D and leads to the loss of functional β-cell mass. Hence, reduction of cell stress and enhancement of β-cell secretory function hold promise towards delayed disease progression and prevention. The SNARE-regulator protein DOC2B plays a crucial role on stimulating the glucose-stimulated insulin secretion (GSIS) and protecting the β-cell from the diabetogenic stresses. As disclosed herein, DOC2B can be developed as a therapeutic target for the treatment of T2D by elucidating the mechanism by which DOC2B preserves functional β-cell mass. Tyrosine phosphorylation of DOC2B's functionally indispensable C2AB domain seems to be required for DOC2B's beneficial function during the pathological conditions related to T2D.

Human and rodent clonal beta-cell lines and primary human islets procured from non-diabetic and T2D donors were used in the experiments. The results showed a marked loss of DOC2B mRNA and protein (˜50% reduction) in the T2D human islets and a resurrected first phase of GSIS in otherwise dysfunctional T2D islets following DOC2B overexpression. Multiple studies confirmed elevated expressions of the CXCL9 and 10 (chemokine receptor 3 ligands) in diabetics. The qPCR and biochemical analysis revealed significant attenuation of cytokine-induced elevation of CXCL9 and 10 gene expressions following DOC2B overexpression in the clonal and primary beta-cells. These results again signified the beneficial effect of DOC2B enrichment in the β-cell. In islet β-cells, glucose-stimulated tyrosine phosphorylation events regulate SNARE complex assembly and GSIS. Using biochemical approaches, DOC2B was determined to be phosphorylated following glucose stimulation and pervanadate (a tyrosine phosphatase inhibitor) treatment in the clonal beta-cells. In silico evaluation from the crystal structure of the C2B domain of DOC2B protein revealed three putative tyrosine phosphorylation sites: Y301 (within the loop region), and Y305, Y309 (within the ordered domain) and the results using point mutants of each putative site (Y to F) indicated the requirement for all three phosphorylation sites for the boosting effect of DOC2B on GSIS in the β-cells. The mutations in at least Y301 and Y305 residues significantly blunted DOC2B's ability to protect β-cells from cytokine-induced beta-cell stress and apoptosis. These results unveil a novel mechanism by which DOC2B enrichment boosts GSIS and protects β-cells against diabetogenic stimuli, the knowledge of which would be crucial for pursuing DOC2B as a novel drug target to prevent and/treat diabetes.

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1: Materials and Methods

Animals: Animals were maintained under protocols approved by the Indiana University Institutional Animal Care and Use Committee and following the National Research Council Guidelines for the Care and Use of Laboratory Animals. Female non-obese diabetic (NOD) NOD/ShiLtJ (RRID:IMSR JAX:001976) and major histocompatibility complex (MHC)-matched control non-obese diabetes resistant (NOR) (RRID:IMSR JAX:002050) mice were obtained from the Jackson Laboratory (Bar Harbor, Maine). Female NOD mice began to convert to T1D at 17-18 weeks of age, with an average conversion rate of 78% by 20 weeks of age, as previously reported [25]. Random blood glucose analysis was performed weekly to monitor conversion to T1D, which is characterized by non-fasting blood glucose levels >250 mg/dl for three consecutive days. To assess DOC2B levels before conversion to T1D, pancreatic islets were isolated, using a method as described previously [26] at 7 weeks (earliest time point for sufficient islet cell yield), 13 weeks (intermediate time point), and 16 weeks of age (latest time point before conversion to T1D). Islet isolation yield decreased in mice less than 8 weeks of age [27]. Islet lysates were then used for SDS-PAGE and immunoblotting. Mouse blood was collected and platelets were isolated as previously described [24]. Platelet lysates were then used for SDS-PAGE and immunoblotting.

Human Subjects: All human studies were conducted in keeping with the principles set out in the Declaration of Helsinki. This protocol was approved by the Indiana University Institutional Review Board. For evaluation of DOC2B levels in human platelets (new-onset T1D study), subjects aged 8-14 (11 males and 6 females) with new-onset T1D were recruited over an 18-month period. Consent was obtained from parents, with assent from the pediatric subjects. Subjects were diagnosed with T1D if they met the criteria of 1 or more positive autoantibodies with clinical features of T1D: hyperglycemia, weight loss, and normal body mass index (BMI) or those who were autoantibody negative but <10 years old at diagnosis. Exclusion criteria were as previously described [17]. Subjects had blood drawn at diagnosis and at the first follow-up appointment 7-10 weeks after diagnosis. Insulin treatment of T1D subjects was started at time of diagnosis. Non-diabetic control subjects (8 males and 6 females) were recruited from the community and matched to T1D subjects based on gender, age, and BMI (see Table 1 for demographic data).

TABLE 1
Pediatric T1D study demographics

| | Non-T1D | | |
|---|---|---|---|
| Characteristic | controls | T1D subjects | |
| Number of subjects | 14 | 17 | |
| Age in years, (range) | 11.4 (8.0-14.3) | 10.3 | (4.3-14.1) |
| Gender (male) | 55% | 57% | |
| BMI (kg/m²)* | 20.2 ± 3.0 | 17.5 ± 2.8 | |
| Number of autoantibodies | — | 0 AutoAb positive: 1 | |
| positive ^{†} | | 1 AutoAb positive: 5 | |
| | | 2 AutoAb positive: 9 | |
| | | 3 AutoAb positive: 2 | |
| Basal insulin requirement | — | 0.30 ± 0.09 | |
| prior to hospital discharge | | | |
| (units/kg/d) | | | |
| C-peptide at diagnosis | — | 110 ± 169 | (13-608) |
| (pmol/l) ^{‡} | | | |
| HbA1c at diagnosis (range) | — | 11.0 ± 1.7% | (7.5 ± 14.2) |
| HbA1c at first follow-up | — | 7.7 ± 0.8% | (6.4-9.0) |

(range)

Abbreviations:
BMI, body mass index;
HbA1c, hemoglobin A1c;
T1D, type one diabetes.
Values displayed are means ± SD unless otherwise noted.
*For BMI calculations, 1 T1D subject did not have a diagnosis height and 1 non-T1D control did not have a registration height. For these subjects, the heights from clinic follow-up were used to calculate BMI.
^{†} The following 3 diabetes-associated antibodies were tested: GAD, miAA, and IA-2A.
^{‡} For C-peptide at diagnosis, n = 13.

Samples were de-identified and coded by the clinical team prior to distribution to the research lab for platelet isolation and analyses. Platelets were isolated by centrifugation from blood, as previously described [28], and lysed for SDS-PAGE and immunoblotting. Upon quantification of the data for each sample, the clinical team re-identified samples to permit grouping of data into T1D vs. non-diabetic for statistical comparisons.

For evaluation of DOC2B levels in human islets (T1D islet transplantation study), samples were obtained from T1D islet transplantation recipients, as approved by the City of Hope Institutional Review Board. Two subjects, aged 43 and 52 years, were recruited for human islet transplantation based on the following criteria: T1D diagnosis with frequent or life-threatening hypoglycemia with or without unawareness symptoms. Blood was obtained from both subjects prior to transplantation (Day 0), and on Day 30 and Day 75 after islet transplantation (see Table 2 for demographic data).

TABLE 2
Baseline adult islet transplant recipient and islet graft characteristics

| Characteristics | COH-027 | COH-028 |
|---|---|---|
| Recipient Characteristics | | |
| Gender | F | M |
| Age at transplant (years) | 43 | 52 |
| Weight (kg) | 76.5 | 92 |
| BMI (kg/m²) | 28.93 | 29.77 |
| Duration of diabetes | 33 | 34 |
| (years) | | |
| HbA1c (%) | 5.5 | 8.5 |
| Insulin intake (units/day) | 28 | 52 |
| Fasting/glucagon- | 0.03/0.02 | <0.02/<0.02 |
| stimulated C-peptide | | |
| (ng/ml) | | |
| Autoantibodies | GAD65-neg \| IA-2-pos | GAD65-pos \| IA-2-neg |
| | mIAA-pos \| ZnT8-neg | mIAA-pos \| ZnT8-neg |
| PRA class I/class II (%) | 0/0 | 0/0 |
| Islet Graft Characteristics | | |
| Total islet dose (IEQ) | 240,133 | 482,755 |
| IEQ/kg | 3,139 | 5,247 |
| Islet purity (%) | 50 | 68 |
| Packed cell volume (ml) | 1.9 | 2.8 |
| Islet viability (%) | 91 | 94 |

Abbreviations:
PRA, panel reactive antibody;
IEQ, islet equivalent.

Platelets were isolated by centrifugation from blood, as previously described [28], and lysed for SDS-PAGE and immunoblotting.

Islet cell transplantation: For the T1D islet transplant study, human pancreata were procured from ABO-compatible, cross-match negative cadaveric donors. The islets were isolated under cGMP conditions by the Southern California Islet Cell Resource Center at City of Hope using a modified Ricordi method. Islets were maintained in culture for up to 72 hours prior to transplantation. Islets were transplanted intraportally with heparinized saline (35 U/kg recipient body weight) using a transhepatic percutaneous approach.

Clinical/laboratory assays: For the new-onset T1D study, autoantibodies to glutamic acid decarboxylase 65 (GAD-65), insulin, and Islet Antigen 2 (IA2) were assayed from peripheral blood at diagnosis at Mayo Medical Laboratories (Rochester, Minnesota). Glycated hemoglobin (HbA1c) was also measured at diagnosis and at first clinic follow-up (7-10 weeks after diagnosis) using the Bayer A1cNow system or the Bayer DCA2000 analyzer (Tarrytown, New York). C-peptide was measured in stored serum samples using the C-peptide ELISA kit (Alpco, Salem, New Hampshire; detection range 20-3000 pM).

For the T1D islet transplant study, plasma C-peptide measurements were performed by the Northwest Lipid Metabolism and Diabetes Laboratory (Seattle, WA) using the Tosoh C-Peptide II Assay (Tosoh Bioscience, Inc, San Francisco, CA; detection range 0.02-30 ng/ml). A fasting C-peptide <0.2 ng/ml and 6-min glucagon-stimulated C-peptide <0.3 ng/ml were used to confirm T1D diagnosis prior to islet transplant. Autoantibodies (GAD-65, IA-2A, insulin [mIAA], and zinc transporter 8 [ZnT8]) were analyzed using radiobinding assays by the Autoantibody/HLA Service Center at the Barbara Davis Center for Diabetes (Aurora, CA).

Ex vivo islet preparations: Non-T1D human cadaveric pancreatic islets were obtained through the Integrated Islet Distribution Program at City of Hope. The islets were prepared and treated with a cytokine mixture (10 ng/ml TNF-α, 100 ng/ml IFN-γ and 5 ng/ml IL-1 β; ProSpec, East Brunswick, NJ, USA) for 72 hours, as previously described [29]. The islets were then used in qRT-PCR analysis or SDS-PAGE followed by immunoblotting.

Extracellular vesicles isolation from human islets: Human islet-derived extracellular vesicles were isolated by sequential centrifugation. Islet conditional medium (PIM(R) 500 mL, Human AB serum 25 mL, PIM(G)L-Glutamine 5 mL, CIPRO 20 ug/mL, Amphotericin B 0.25 ug/mL and Gentamicin 20 μg/mL) was centrifuged at 500×g for 10 minutes to remove dead cells. The supernatant was transferred to high-speed centrifuge tubes and was centrifuged at 12,500×g for 25 minutes to remove cell debris from the conditional medium. Following centrifugation, the supernatant was transferred to the 70 mL ultracentrifugation tubes and was spun at 110,000×g for 70 minutes. After removing the supernatant, pellets were washed, resuspended in 70 mL of PBS, and was centrifuged at 110,000×g for 70 minutes. EV samples were resuspended in PBS and stored at −80° C. until use.

Polyclonal antibody production for immunoassays: As commercial antibodies have cross-reactivity with β-actin and the DOC2A isoform, human DOC2B custom antibodies were generated. To select specific regions (79-99, 96-116, 249-267, 23-116) of DOC2B amino acid for antibody epitopes, previous published protocol was followed (Fukuda et al., 2009 and Groffen et al., 2010), and online epitope design tools (hpcwebapps.cit.nih.gov/AbDesigner/, www.abcam.com/protocols/tips-for-designing-a-good-peptide-immunogen), predictive algorithms, 3D structure by Pymol, and IUPred were used. Four different peptides (Pacific Immunology, Ramona, CA) were synthesized and a single terminal Cysteine was added to allow for conjugation to the carrier protein. This process represents the optimal conjugation chemistry and allows all epitopes within the peptide to be freely exposed. Each peptide immunized two rabbits, and antibodies were purified by affinity column (Pacific Immunology, Ramona, CA, USA).

Cell culture and transient transfection: To specify detection of DOC2B, not DOC2A using purified DOC2B antibodies, mouse MIN6 β-cells were cultured in Dulbecco's modified Eagle's medium (DMEM) as previous described (Ke et al., 2007) and transfected with DOC2A-EGFP or pcDNA3 plasmid DNA using the lipofectamine reagent. Forty-eight hours after transfection, the cells were lysed with 1% Nonidet P-40 lysis buffer (25 mM HEPES pH 7.4, 1% Nonidet P-40, 10% glycerol, 137 mM NaCl, 1 mM sodium vanadate, 50 mM NaF, 10 mM NaPP, 10 μg/ml aprotinin, 5 μg/ml leupeptin, 1 μg/ml pepstatin, and 1 mM phenylmethylsulfonyl fluoride).

Indirect ELISA: A high binding 96-well plate (Cat #762071, Greiner Bio-One, Kremsmünster, Austria) was coated with 100 μL of recombinant human DOC2B in coating buffer (Bio-Rad) and incubated over-night in room temperature. The plates were then washed in 0.05% Tween20 (Fisher bioreagents) in PBS and blocked with blocking buffer (Bio-Rad) for 2.5 hours in room temperature. The plates were washed, and primary DOC2B antibodies was added at a 1:20000 dilutions and incubated over-night in 4° C. After washing, secondary antibody (1:5000) was added for 1 hour in 37° C., washed and TMB peroxidase (Bio-Rad) was added for 10 minutes. The reaction was stopped by adding 0.25M sulphuric acid. The plate was spectrophotometrically read with SYNERGY HTX plate reader in a single wavelength mode at 450 nm and analyzed in PRISM software using a 4-parameter algorithm to generate a standard curve.

Immunofluorescence: Human paraffin-embedded pancreatic tissue sections were obtained from the Network for Pancreatic Organ Donors with Diabetes (nPOD). Five sections from formalin-fixed paraffin-embedded (FFPE) tissue samples were obtained from T1D (n=3) and age and BMI-matched non-diabetic (n=3) donors. Pancreas sections were immunostained with primary and secondary antibodies listed in Table 3.

TABLE 3
Primary and secondary antibodies used in study

| | | | |
|---|---|---|---|
| Protein Target | Source | Catalogue No. | RRID No. |

Primary antibodies used in NOD mouse study/ex vivo stet study

| | | | |
|---|---|---|---|
| DOC2B | Proteintech | 20574-1-AP | AB_10696316 |
| Tubulin | Abcam | ab56676 | AB_945996 |
| iNOS | Millipore | ABN26 | AB_10805939 |

Primary antibodies used in New-onset T1D/T1D transplant study

| | | | |
|---|---|---|---|
| DOC2B | Abnova | H00008447-B01P | AB_10549446 |
| GAPDH | Abnova | ab9485 | AB_307275 |

Primary antibodies used in immunofluorescence study

| | | | |
|---|---|---|---|
| DOC2B | Proteintech | 20574-1-AP | AB_10696316 |
| Insulin | Abcam | ab7842) | AB_306130 |

Secondary antibodies

| | | | |
|---|---|---|---|
| goat anti-rabbit | Bio-Rad | 1706515 | AB_11125142 |
| goat anti-mouse | Bio-Rad | 1706516 | AB_11125547 |
| Alexa Fluor 568 | Abcam | ab175471 | AB_2576207 |
| goat anti-rabbit | | | |
| Alexa Fluor 488 | Thermo | A-11073 | AB_2534117 |
| goat anti-guinea pig | | | |

Slides were counterstained to mark the nuclei, using 4′,6-diamidino-2-phenylindole (DAPI) (Vectashield; Vector Laboratories, Burlingame, CA) and viewed using a Keyence BZ X-700 fluorescence microscope (Keyence Corporation, Itasca, IL). All human T1D samples were prepared and processed at the same time; confocal images were taken with identical acquisition settings. Islet immunofluorescence was assessed by imaging 20-30 islets (grouping of four or more insulin-positive cells) per subject. Analysis was performed in a blinded fashion using Image-Pro Software (Media Cybernetics, Rockville, MD, USA) to quantify fluorescence intensities using methods as previously described [30]. Defined regions of interest (ROIs) were used to delimit islets from adjacent acinar tissue and average intensity measurements of insulin and DOC21B were quantified by splitting the merged image into two color channels with the same ROL.

Immunoblotting: Platelet and islet protein lysates for the NOD mouse study were resolved on a 10% SDS-PAGE gel and transferred to standard PVDF (Bio-Rad, Hercules, CA, USA). Platelet proteins from the new-onset T1D study were resolved on a 10% SDS-PAGE gel using an SE400 air-cooled 18×16 cm vertical protein electrophoresis unit (Hoefer, Inc. Holliston, MA) and transferred to standard PVDF (Bio-Rad). Platelet proteins from the T1D islet transplant study were resolved on a 12% SDS-PAGE gel using a Criterion™ 13.3×8.7 cm vertical electrophoresis unit (Bio-Rad) and transferred to standard PVDF. All blots were probed as outlined in Table 3.

Samples (10 μg of proteins) were resolved on 15% SDS-PAGE gel, and then transferred onto a 0.45 μM polyvinylidene difluoride membrane (PVDF; Bio-Rad, Hercules, CA, USA). After that, the PVDF membranes were blocked with 5% non-fat milk in TBST at room temperature for 1 hour. The membranes were briefly washed with TBST and then incubated overnight at 4° C. on an orbital shaker with the following primary antibodies in 1% bovine serum albumin (BSA) in TBST with 0.02% sodium azide: anti-DOC2B #1 (1:2000, home-made), anti-DOC2B #2 (1:2000, home-made), anti-DOC2B #3 (1:2000, home-made), anti-DOC2B #4 (1:2000, home-made), anti-EGFP (1:1000, Cat #632381, Takara, Mountain View, CA, USA) and anti-Transferrin (1:1000, Cat #109503, Abcam, Cambridge, UK). Following this, the membranes were washed three times with TBST for 5 minutes and incubated with the following secondary antibodies in 5% non-fat milk in TBST for 1 hour at room temperature: goat-anti-rabbit IgG (HL) conjugated to horseradish peroxidase (HRP) (1:5000, Cat #172-1019, Bio-Rad, Hercules, CA, USA) or goat anti-mouse IgG (HL)-HRP (1:5000, Cat #172-1011, Bio-Rad, Hercules, CA, USA). Following the secondary antibody, the membranes were washed three times with TBST for 5 minutes. Protein bands were visualized by either ECL or ECL prime (GE Healthcare, Chicago, IL, USA) and detected by Chemidoc touch (Bio-Rad, Hercules, CA, USA).

Quantitative real-time PCR: Total RNA was isolated from human islets using the Qiagen RNeasy Plus Mini Kit (Qiagen, Valencia, CA, USA) and assessed using the QuantiTect SYBR Green RT-PCR kit (Qiagen). Primers used for the detection of hDoc2b are as follows: forward: 5′-CCAGTAAGGCAAATAAGCTC-3′ (SEQ ID NO: 15) and reverse: 5′-GGGTTTCAGCTTCTTCA-3′ (SEQ ID NO: 16). Standard tubulin primers (Cat: QT00089775, Qiagen) were used for normalization.

Statistical analysis: Data were evaluated for statistical significance using Student's t test for comparison of two groups; ANOVA and Tukey's post-hoc tests (GraphPad Software, La Jolla, CA, USA) were used for comparison of more than two groups. Data are expressed as the average±SEM. Statistical analysis was performed using Graphpad Prism 8. Unpaired Student's t-test was performed to compare T1D with normal. Significance was considered at P<0.05.

### Example 2: Low DOC2B Levels in Pre-Diabetic NOD Mouse Platelets and Islets

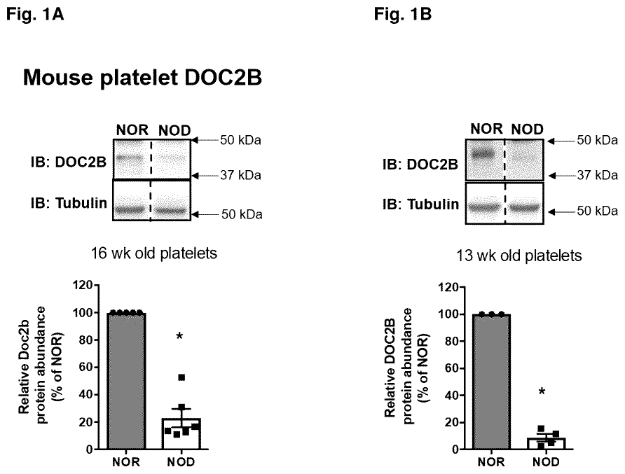

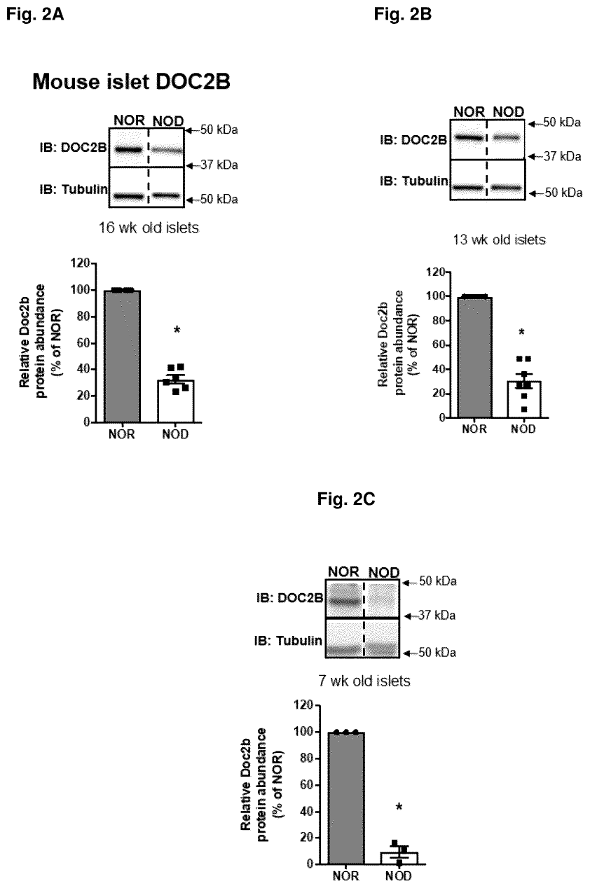

To investigate whether DOC2B protein levels are altered in the blood prior to onset of T1D, platelet DOC2B abundance in young pre-diabetic NOD mice and MHC-matched NOR mice was examined. Immunoblotting revealed that platelets from 16- and 13-week old NOD mice exhibited up to a 90% reduction in DOC2B protein levels (FIG. 1) compared to NOR platelets. Furthermore, islets from 16- and 13-week old NOD mice showed at least a 65% reduction in DOC2B protein levels (FIG. 2) compared to NOR islets. NOD islets from as early as 7 weeks of age showed a 90% reduction in DOC2B protein (FIG. 2). The average blood glucose levels from random blood testing of NOD and NOR mice were below 250 mg/dL at 7, 13, and 16 weeks (Table 4), indicating that the mice had not yet converted to diabetes. These data show that DOC2B protein abundance is reduced in both islets and platelets of prediabetic mice.

TABLE 4
Average blood glucose levels of NOD and NOR
mice at 16, 13, and 7 weeks
Avg. Non-fasting Blood Glucose (mg/dL)

| | 16 weeks | 13 weeks | 7 weeks |
|---|---|---|---|
| NOR | 197 ± 17 | 183 ± 10 | 130 ± 6 |
| NOD | 196 ± 19 | 194 ± 22 | 127 ± 5 |

Data represent the average ± S.E; n = 6 per group for mice at 16 and 13 weeks; n = 5 per group at 7 weeks. Random non-fasting blood glucose was measured for NOR and NOD female mice at 13 and 16 weeks of age. No statistical differences were seen.

### Example 3: Low DOC2B Levels in New-Onset T1D Human Platelets

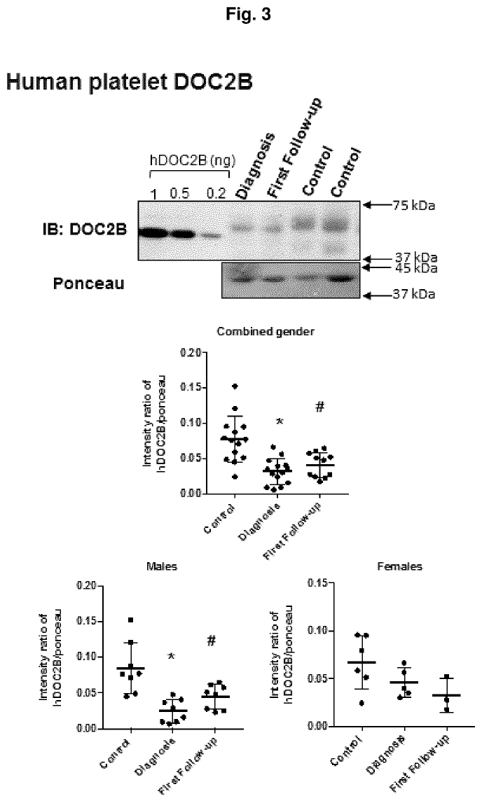

In the new-onset T1D study, the protein content of DOC2B was quantified using platelets from new-onset T1D subjects in comparison to controls (Table 1). Platelets from new-onset T1D subjects exhibited reduced protein levels of DOC2B for both genders, both at diagnosis and at first clinic follow-up 7-10 weeks later. When males and females were assessed separately, DOC2B levels were reduced in males by ˜70% compared to non-diabetic control subjects, persisting even after insulin treatment of the patient and reduction of HbA1c (FIG. 3). The significant loss of DOC2B at T1D diagnosis was selective for DOC2B compared to another exocytosis protein, syntaxin 4 (STX4) (FIG. 7). These data indicate that DOC2B was decreased in T1D platelets independent of glycemic control, relative to non-diabetic human platelets, and that platelet DOC2B levels were already diminished at T1D diagnosis.

### Example 4: Ex Vivo Pro-Inflammatory Cytokines Treatment Reduces Human Islet DOC2B Levels

T1D is associated with elevated circulating pro-inflammatory cytokines, which damages β-cells [31]. Because obtaining pancreatic islets from living T1D subjects is virtually impossible, the relationship between T1D and DOC2B levels was evaluated by treating human cadaveric non-diabetic islets (Table 5) ex vivo with pro-inflammatory cytokines in effort to simulate the circulating milieu.

TABLE 5
Non-diabetic human islet donor characteristics

| Unos/ | | | | | | Islet | |
|---|---|---|---|---|---|---|---|
| COH | | | | | Islet | Viabil- | Exp. |
| ID no. | Sex | Age | BMI | Race | Purity | ity | Use |
| ACIN402 | M | 49 | 40.0 | Hispanic | N/A | N/A | protein |
| ACIY103 | M | 24 | 24.6 | Caucasian | 78% | N/A | protein |
| Hu966 | M | 20 | 30.6 | African | 88% | N/A | protein |
| | | | | American | | | |
| ADBL | F | 53 | 23.8 | Caucasian | 90% | 90% | protein |
| ADFE489 | F | 45 | 23.1 | Asian | N/A | N/A | mRNA |
| ADDV480 | M | 52 | 25.4 | Caucasian | N/A | N/A | mRNA |
| AEFU443 | F | 49 | 29 | Caucasian | 95% | 95% | mRNA |
| Hu1000 | M | 49 | 29 | Hispanic | N/A | N/A | mRNA |
| Hu966 | M | 20 | 30.6 | African | 88% | N/A | mRNA |
| | | | | American | | | |
| ACIY103 | M | 24 | 24.6 | Caucasian | 78% | N/A | mRNA |

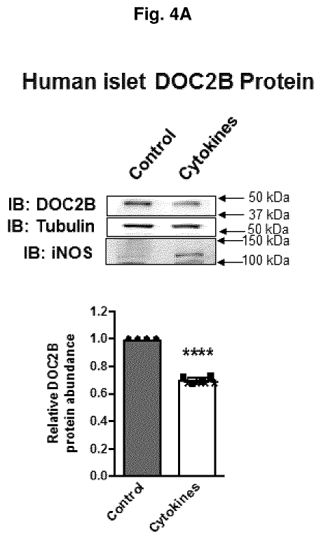

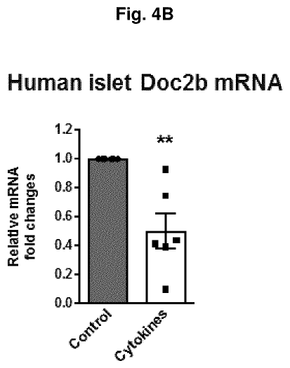

Cytokine treatment (IL-1β, TNF-αL, INF-γ) elevated the levels of islet iNOS, consistent with the reported effects of cytokine exposure [32]. Correspondingly, DOC21B protein and mRNA levels were reduced by 30% and 50%, respectively (FIGS. 4A-4B). These data suggest that a T1D-like milieu can decrease DOC21B levels in human islets.

### Example 5: Reduced DOC2B Protein in Human Early-Onset T1D Islets and Pre-T2D

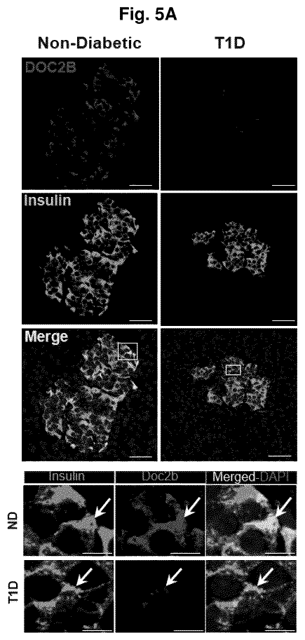

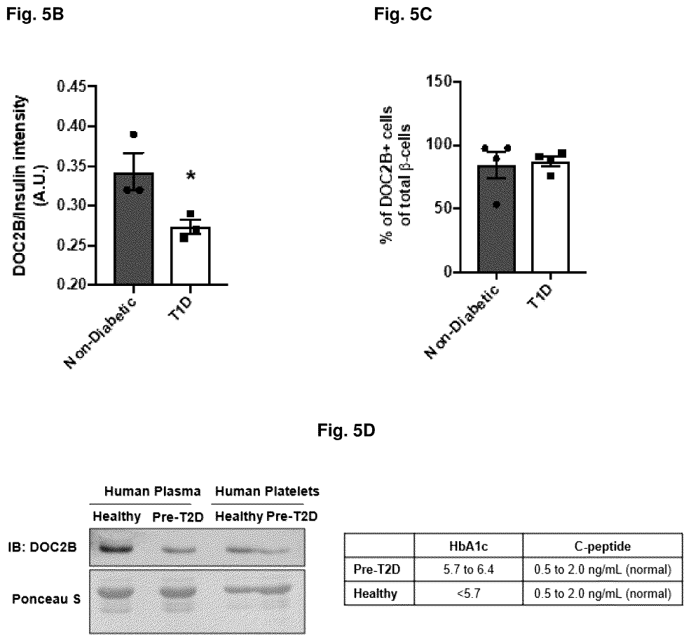

To investigate changes in DOC21B levels in T1D human pancreata, paraffin embedded slides (obtained from nPOD) from cadaveric donors were used for DOC21B immunofluorescence evaluation in early-onset pediatric T1D (5 years or less with T1D) (n=3) versus matched controls (n=3) (FIG. 5A, and Table 6).

TABLE 6
nPOD sample human pancreata donor characteristics

| nPOD ID no. | Sex | Age | BMI | Race | Years of T1D |
|---|---|---|---|---|---|
| 6113 | F | 13.1 | 24.7 | Caucasian | 1.5 |
| 6342 | F | 14 | 24.3 | Caucasian | 2 |
| 6243 | M | 13 | 21.3 | Caucasian | 5 |
| 6386 | M | 14 | 23.9 | Caucasian | ND |
| 6392 | M | 14.1 | 23.6 | Caucasian | ND |
| 6340 | M | 9.7 | 20.3 | Caucasian | ND |

Abbreviations:
ND; non-diabetic.

By measuring relative immunofluorescent intensities, a decrease in DOC2B abundance in T1D islets versus that in non-diabetic controls was detected (FIG. 5B). Although the relative number of DOC2B-positive β-cells in non-diabetic and T1D islets were similar (FIG. 5C), DOC2B intensity was reduced in T1D β-cells. FIG. 5D shows a reduction in pre-type 2 diabetes. FIG. 5 demonstrates that DOC2B is a validated pan-diabetes biomarker.

### Example 6: DOC2B Levels are Restored after Clinical Islet Transplantation

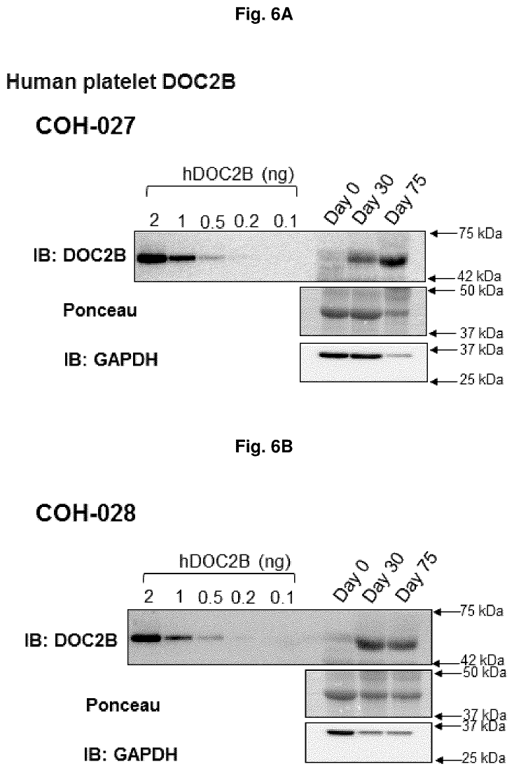

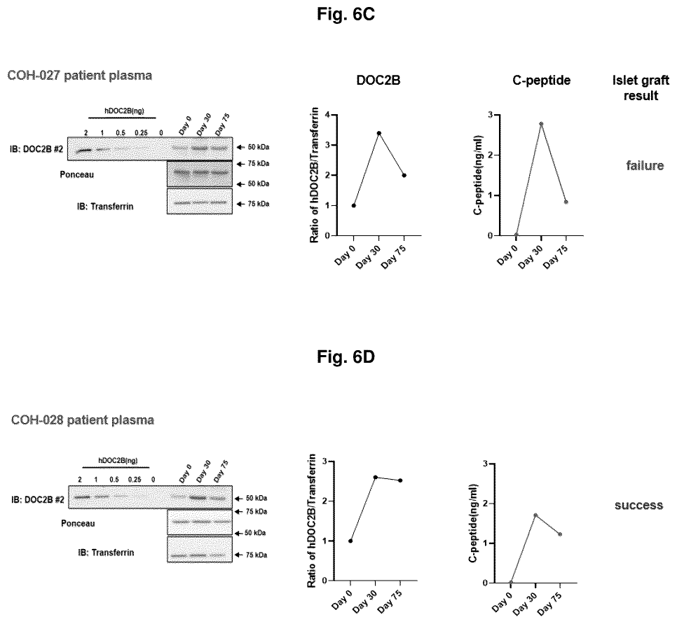

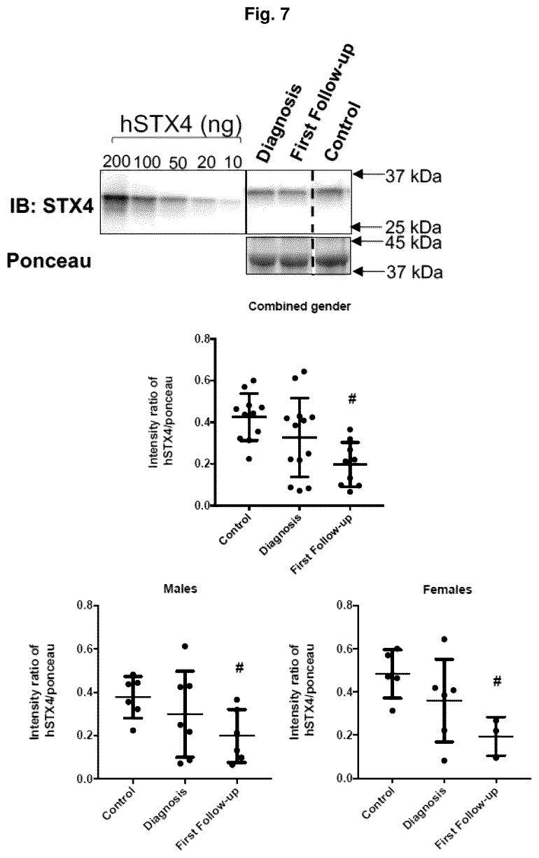

In the T1D islet transplantation study (Table 2), the pre-transplant platelet DOC2B levels were very low in both subjects relative to an hDOC2B protein standard curve (FIGS. 6A-6B, Day 0). Notably, within 30 days of transplantation, each T1D islet recipient showed a robust increase in platelet DOC2B protein, which persisted to 75 days after transplantation (FIGS. 6A-6B, Days 30 and 75). FIGS. 6C and 6D show that the plasma DOC2B levels were remediated following islet transplant in the same two adult T1D DOC2B deficient patients. These data coincide with changes in C-peptide levels in these subjects: while each subject had low to almost undetectable fasting/glucagon-stimulated C-peptide levels before transplantation, the C-peptide levels were substantially increased by 30 days after transplantation (Table 7). As C-peptide levels are indicative of overall islet function, these data suggest that in humans, DOC2B levels in platelets correlate with relative functional β-cell mass.

TABLE 7
Islet transplant recipient treatment and outcome summary

| | | islet Transplant Recipients | |
|---|---|---|---|
| | | COH-027 | COH-028 |
| Immunosuppression Regimen | | Induction: | Induction: |
| | | rATG, etanercept, anakinra | rATG, etanercept, anakinra, |
| | | Maintenance: | Maintenance: |
| | | tacrolimus, MMF, +/−sirolimus | tacrolimus, MMF +/−sirolimus |
| Additional Immunosuppression for | | Solumedrol, Plasmapheresis, IVig & | NA |
| suspected islet graft rejection | | Rituxan for suspected islet graft | |
| | | rejection | |
| HbA1c (%) | Pre-Tx | 5.5 | 8.5 |
| | Day 30 | ND | ND |
| | Day 75 | 5.3 | 6 |
| Insulin Intake (units/day) | Pre-Tx | 28 | 52 |
| | Day 30 | 13 | 28 |
| | Day 75 | 15 | 8 |
| Fasting/Glucagon-Stimulated | Pre-Tx | 0.03/0.02 | <0.02/<0.02 |
| C-peptide (ng/ml) | Day 30 | 2.78/3.56 | 1.71/3.39 |
| | Day 75 | 0.8.4/1.42 | 12.3/2.40 |
| Mixed Meal Tolerance Test (MMTT) | Pre-Tx | ND | ND |
| C-peptide at 0/90 min | Day 30 | ND | ND |
| | Day 75 | 0.63/2.65 | 1.62/2.95 |
| Oral Glucose Tolerance Test (OGTT) | Pre-Tx | ND | ND |
| BG (mg/dl) \| C-pep (ng/ml, | Day 30 | BG: 102/197 \| C-pep: 1.49/6.72 | BG: 127/228 \| C-p: 1.65/4.21 |
| Fasting/120 min | Day 75 | BG: 101/199 \| C-pep: 0.71/3.52 | BG: 148/310 \| C-p: 1.46/3.22 |
| Autoantibodies | Pre-Tx | GAD65-neg/IA-2-pos | GAD65-pos/IA-2-neg |
| | | mIAA-pos/ZnT8-neg | rnIAA-pos/ZnT8-neg |
| | Day 75 | GAD65-pos*/IA-2-pos | GAD65-pos / IA-2-neg |

| | |
|---|---|
| mIAA-neg*/ZnT8-neg | mIAA-pos/ZnT8-neg |

NA = Not applicable;
ND = Not done;
Pre-TX: Pre-Transpiant (baseline);
*Denotes change in auto or allo-antibodies status from baseline

### Example 7: DOC2B Antibodies Test Results

Four anti-DOC2B antibodies were developed: Antibody #1 binds to human DOC2B amino acid sequence AA 79-99, Antibody #2 binds to human DOC2B amino acid sequence AA 96-116, Antibody #3 binds to human DOC2B amino acid sequence AA 249-267 for detection of C2AB, and Antibody #4 binds to human DOC2B amino acid sequence AA 23-62, 55-92, and 82-116. Table 8 below shows the immunoblotting results.

TABLE 8
Immunoblotting

| | | | | | | | | | | IF: | IF: |
|---|---|---|---|---|---|---|---|---|---|---|---|
| | | recomb | human | | C2AB | Human | | human | IF: | Mouse | Human |
| Antigen | Ab | Doc2b | EndoC bH1 | MIN6 | domain | Platelet | Doc2a | plasma | MIN6 | pancreas | pancreas |
| 79-99 | #1 | +++ | ? | ? | ND | ? | ND | ? | ND | ND | NA |
| 96-116 | #2 | ++ | +++ | +++ | ND | +++ | ND | ? | ++ | + | + |
| 249-267 | #3 | + | ++ | ++ | +++ | ++ | detects | +++ | ++ | ++ | ++ |
| 23-116 | #4 | +++ | ++ | ++ | ? | ? | detects | ? | ++ | ? (BP) | ? (BP) |

Based on 1:1000 dilution; ND: not detected; NA: not applicable; ?: multiple bands; (BP): competitive binding peptide test not yet processed.

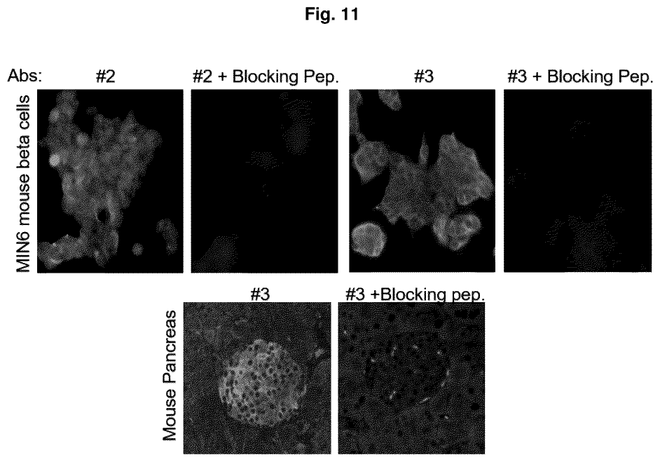

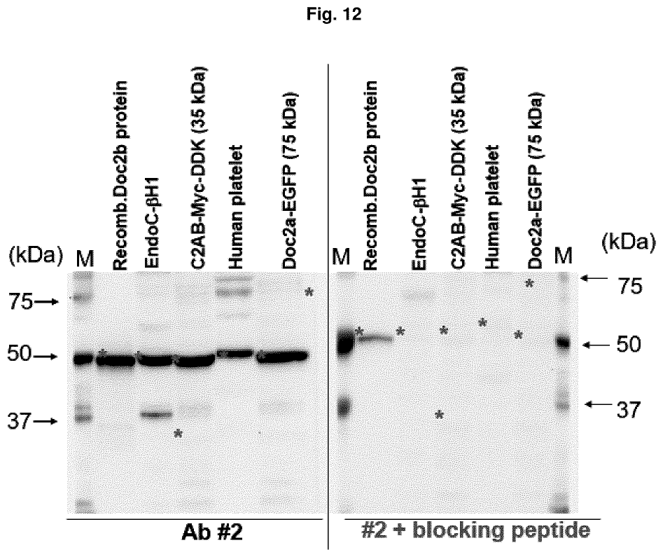

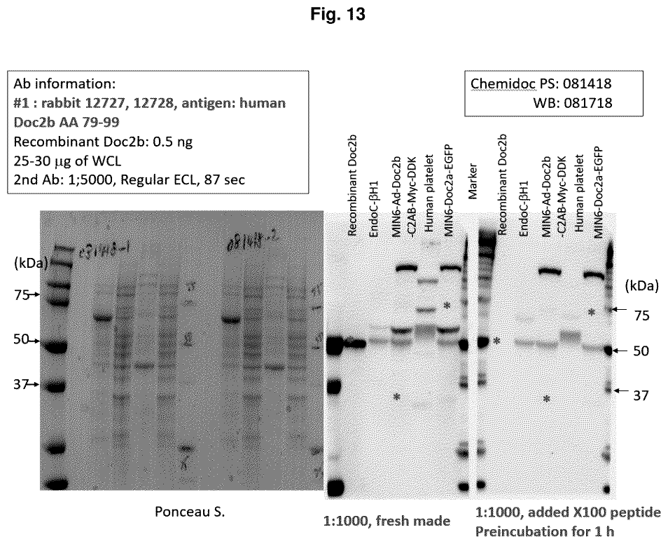

FIG. 11 shows that the antibodies disclosed herein can be detected in β cells by immunofluorescent detection. FIGS. 12 and 13 show the immunoblot detection of DOC2B Antibody #2. In FIG. 12, affinity purified Ab #2 was used at 1,000 dilution to detect endogenous DOC2B present in a variety of cell lysates. Each lane of the 10% SDS-PAGE was loaded with 25-30 mg of cell lysates indicated, proteins resolved were transferred to PVDF and used for immunoblot. Following 1 h incubation with Ab #2 at 1,000 dilution, the PVDF was washed three times with TBS-Tween for a total of 30 min at RT, then probed with a secondary antibody at a dilution of 1:5,000 for 1 h RT, and detection of bands using enhanced chemiluminescence (ECL, 45 sec exposure shown). In FIG. 13, affinity purified Ab #3 was used similarly to that of Ab #2, the only other difference being ECL detection for 87 sec.

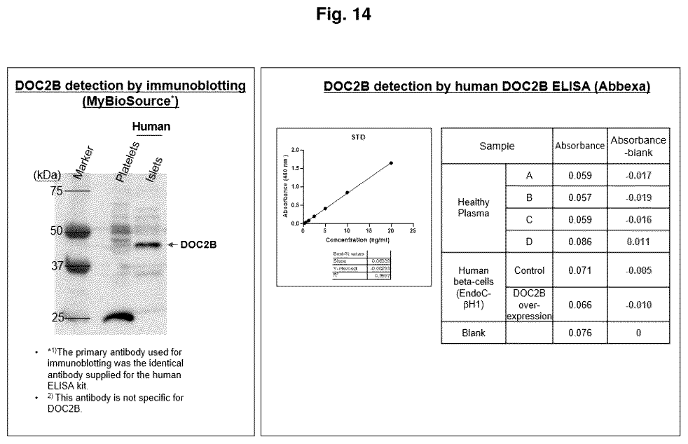

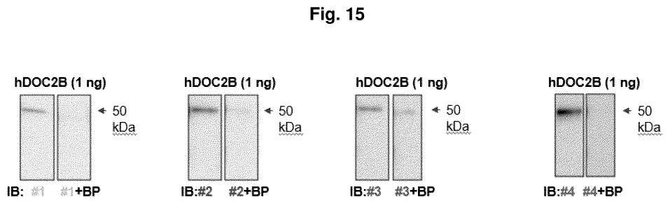

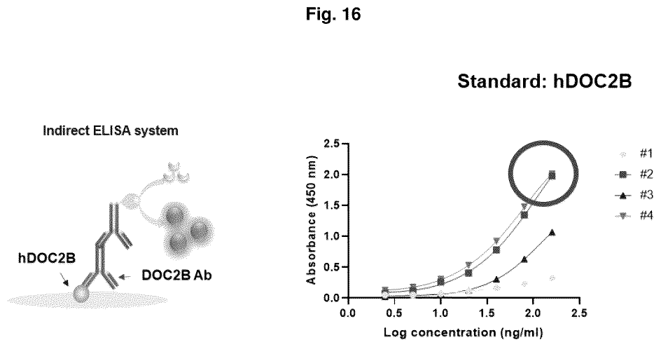

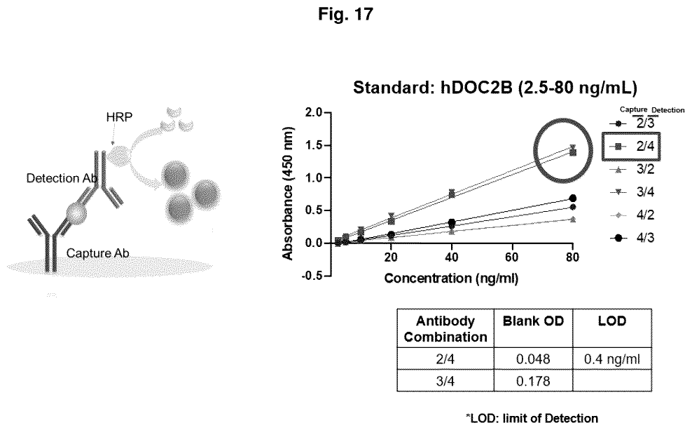

FIG. 14 shows that commercially available human DOC2B ELISAs are not suitable for detecting DOC2B. FIG. 15 shows validation of custom DOC2B Antibodies #1, #2, #3, and #4 in a denatured form. All custom rabbit polyclonal DOC2B antibodies detected a single DOC2B protein band of the appropriate molecular weight under denaturing condition. *BP: blocking peptide. FIG. 16 shows that custom rabbit anti-human DOC2B antibodies recognized the native form of human recombinant DOC2B. FIG. 17 shows that sandwich ELISA was developed using custom rabbit polyclonal anti-human DOC2B antibodies.

### Example 8: Extracellular Vesicles from Human Islets

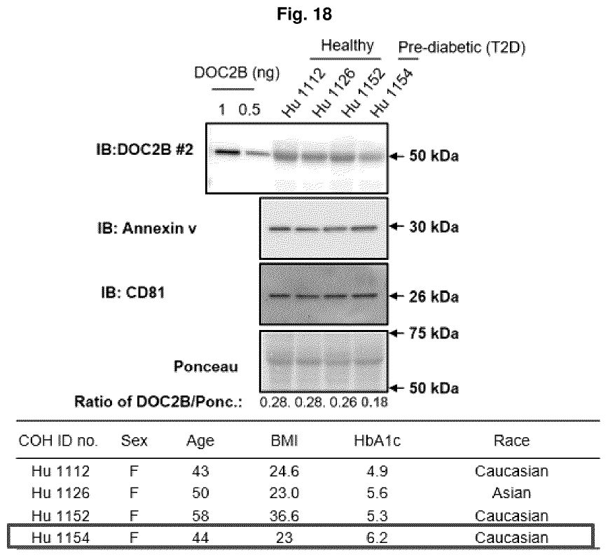

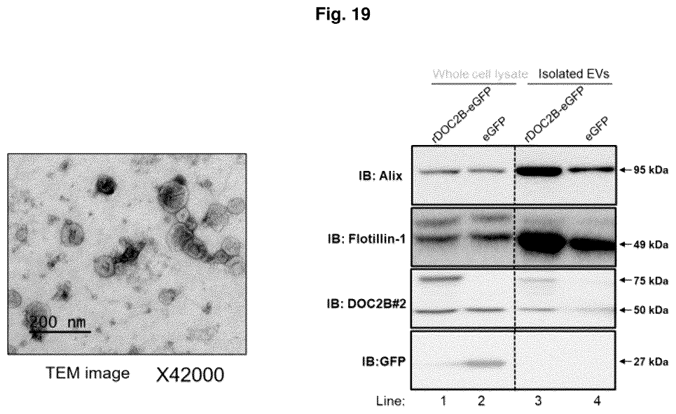

To determine an association DOC2B levels between in plasma and human islets, the EV isolated from human islets supernatant was examined to see if it contained DOC2B proteins. Immunoblotting revealed that the islet EVs isolated from the supernatant of age-matched four donors contain DOC2B proteins (FIG. 18), indicating that DOC2B in plasma stems from the islets. Of four donors, three (Hu1112, Hu1126, and Hu1152) are healthy (HbA1c<5.7), and one (Hu1154) is prediabetic (HbA1c: 6.2). DOC2B levels in a prediabetic donor were lower than those in healthy donors (FIG. 18). These data suggest that DOC2B protein abundance in plasma may be reflective of the progression of T1D. FIG. 19 shows that DOC2B-laden EVs were predominantly released by β-cells.

As stated above, the foregoing is merely intended to illustrate the various embodiments of the present invention. As such, the specific modifications discussed above are not to be construed as limitations on the scope of the invention. It will be apparent to one skilled in the art that various equivalents, changes, and modifications may be made without departing from the scope of the invention, and it is understood that such equivalent embodiments are to be included herein. All references cited herein are incorporated by reference as if fully set forth herein.

### REFERENCES

1. Ferrannini E, Mari A, Nofrate V, Sosenko J M, Skyler J S. Progression to diabetes in relatives of type 1 diabetic patients: mechanisms and mode of onset. Diabetes. 2010; 59:679-685.
2. Siljander H T, Hermann R, Hekkala A, Lahde J, Tanner L, Keskinen P, Ilonen J, Simell O, Veijola R, Knip M. Insulin secretion and sensitivity in the prediction of type 1 diabetes in children with advanced beta-cell autoimmunity. Eur J Endocrinol. 2013; 169:479-485.
3. Keymeulen B, Vandemeulebroucke E, Ziegler A G, Mathieu C, Kaufman L, Hale G, Gorus F, Goldman M, Walter M, Candon S, Schandene L, Crenier L, De Block C, Seigneurin J M, De Pauw P, Pierard D, Weets I, Rebello P, Bird P, Berrie E, Frewin M, Waldmann H, Bach J F, Pipeleers D, Chatenoud L. Insulin needs after CD3-antibody therapy in new-onset type 1 diabetes. N Engl J Med. 2005; 352:2598-2608.
4. Herold K C, Gitelman S E, Willi S M, Gottlieb P A, Waldron-Lynch F, Devine L, Sherr J, Rosenthal S M, Adi S, Jalaludin M Y, Michels A W, Dziura J, Bluestone J A. Teplizumab treatment may improve C-peptide responses in participants with type 1 diabetes after the new-onset period: a randomised controlled trial. Diabetologia. 2013; 56:391-400.
5. Gaisano H Y. Recent new insights into the role of SNARE and associated proteins in insulin granule exocytosis. Diabetes Obes Metab. 2017; 19 Suppl 1:115-123.
6. Kiraly-Borri C E, Morgan A, Burgoyne R D, Weller U, Wollheim C B, Lang J. Soluble N-ethylmaleimide-sensitive-factor attachment protein and N-ethylmaleimide-insensitive factors are required for Ca2+-stimulated exocytosis of insulin. Biochem J. 1996; 314:199-203.
7. Miyazaki M, Emoto M, Fukuda N, Hatanaka M, Taguchi A, Miyamoto S, Tanizawa Y. DOC2b is a SNARE regulator of glucose-stimulated delayed insulin secretion. Biochem Biophys Res Commun. 2009; 384:461-465.
8. Ke B, Oh E, Thurmond D C. Doc2beta is a novel Munc18c-interacting partner and positive effector of syntaxin 4-mediated exocytosis. J Biol Chem. 2007; 282:21786-21797.
9. Ramalingam L, Oh E, Yoder S M, Brozinick J T, Kalwat M A, Groffen A J, Verhage M, Thurmond D C. Doc2b is a key effector of insulin secretion and skeletal muscle insulin sensitivity. Diabetes. 2012; 61:2424-2432.
10. Li J, Cantley J, Burchfield J G, Meoli C C, Stockli J, Whitworth P T, Pant H, Chaudhuri R, Groffen A J, Verhage M, James D E. DOC2 isoforms play dual roles in insulin secretion and insulin-stimulated glucose uptake. Diabetologia. 2014; 57:2173-2182.
11. Ramalingam L, Oh E, Thurmond D C. Doc2b enrichment enhances glucose homeostasis in mice via potentiation of insulin secretion and peripheral insulin sensitivity. Diabetologia. 2014; 57:1476-1484.
12. Keller M P, Choi Y, Wang P, Davis D B, Rabaglia M E, Oler A T, Stapleton D S, Argmann C, Schueler K L, Edwards S, Steinberg H A, Chaibub Neto E, Kleinhanz R, Turner S, Hellerstein M K, Schadt E E, Yandell B S, Kendziorski C, Attie A D. A gene expression network model of type 2 diabetes links cell cycle regulation in islets with diabetes susceptibility. Genome Res. 2008; 18:706-716.
13. Ziegler A G, Rewers M, Simell O, Simell T, Lempainen J, Steck A, Winkler C, Ilonen J, Veijola R, Knip M, Bonifacio E, Eisenbarth G S. Seroconversion to multiple islet autoantibodies and risk of progression to diabetes in children. Jama. 2013; 309:2473-2479.
14. Sosenko J M, Krischer J P, Palmer J P, Mahon J, Cowie C, Greenbaum C J, Cuthbertson D, Lachin J M, Skyler J S. A risk score for type 1 diabetes derived from autoantibody-positive participants in the diabetes prevention trial-type 1. Diabetes Care. 2008; 31:528-533.
15. Insel R A, Dunne J L, Atkinson M A, Chiang J L, Dabelea D, Gottlieb P A, Greenbaum C J, Herold K C, Krischer J P, Lernmark A, Ratner R E, Rewers M J, Schatz D A, Skyler J S, Sosenko J M, Ziegler A G. Staging presymptomatic type 1 diabetes: a scientific statement of JDRF, the Endocrine Society, and the American Diabetes Association. Diabetes Care. 2015; 38:1964-1974.
16. Sims E K, Chaudhry Z, Watkins R, Syed F, Blum J, Ouyang F, Perkins S M, Mirmira R G, Sosenko J, DiMeglio L A, Evans-Molina C. Elevations in the Fasting Serum Proinsulin-to-C-Peptide Ratio Precede the Onset of Type 1 Diabetes. Diabetes Care. 2016; 39:1519-1526.
17. Watkins R A, Evans-Molina C, Terrell J K, Day K H, Guindon L, Restrepo I A, Mirmira R G, Blum J S, DiMeglio L A. Proinsulin and heat shock protein 90 as biomarkers of beta-cell stress in the early period after onset of type 1 diabetes. Transl Res. 2016; 168:96-106.e101.
18. Fisher M M, Watkins R A, Blum J, Evans-Molina C, Chalasani N, DiMeglio L A, Mather K J, Tersey S A, Mirmira R G. Elevations in Circulating Methylated and Unmethylated Preproinsulin DNA in New-Onset Type 1 Diabetes. Diabetes. 2015; 64:3867-3872.
19. Tang K, Hynan L S, Baskin F, Rosenberg R N. Platelet amyloid precursor protein processing: a bio-marker for Alzheimer's disease. J Neurol Sci. 2006; 240:53-58.
20. Peterson J E, Zurakowski D, Italiano J E, Jr., Michel L V, Connors S, Oenick M, D'Amato R J, Klement G L, Folkman J. VEGF, PF4 and PDGF are elevated in platelets of colorectal cancer patients. Angiogenesis. 2012; 15:265-273.
21. Zheng Y, Wang L, Zhu Z, Yan X, Zhang L, Xu P, Luo D. Altered platelet calsequestrin abundance, Na(+)/Ca(2)(+) exchange and Ca(2)(+) signaling responses with the progression of diabetes mellitus. Thromb Res. 2014; 134:674-681.
22. Witas H W, Rozalski M, Watala C, Mlynarski W, Sychowski R, Bodalski J. Do determinants of platelet function co-segregate with genetic markers of type 1 diabetes mellitus?Platelets. 1999; 10:169-177.
23. Malachowska B, Tomasik B, Szadkowska A, Baranowska-Jazwiecka A, Wegner O, Mlynarski W, Fendler W. Altered platelets' morphological parameters in children with type 1 diabetes—a case-control study. BMC Endocr Disord. 2015; 15:17.
24. Ye S, Karim Z A, Al Hawas R, Pessin J E, Filipovich A H, Whiteheart S W. Syntaxin-11, but not syntaxin-2 or syntaxin-4, is required for platelet secretion. Blood. 2012; 120:2484-2492.
25. Tersey S A, Nishiki Y, Templin A T, Cabrera S M, Stull N D, Colvin S C, Evans-Molina C, Rickus J L, Maier B, Mirmira R G. Islet beta-cell endoplasmic reticulum stress precedes the onset of type 1 diabetes in the nonobese diabetic mouse model. Diabetes. 2012; 61:818-827.
26. Oh E, Stull N D, Mirmira R G, Thurmond D C. Syntaxin 4 up-regulation increases efficiency of insulin release in pancreatic islets from humans with and without type 2 diabetes mellitus. J Clin Endocrinol Metab. 2014; 99:E866-870.
27. Stull N D, Breite A, McCarthy R, Tersey S A, Mirmira R G. Mouse islet of Langerhans isolation using a combination of purified collagenase and neutral protease. J Vis Exp. 2012.
28. Reuwer A Q, Nieuwland R, Fernandez I, Goffin V, van Tiel C M, Schaap M C, Berckmans R J, Kastelein J J, Twickler M T. Prolactin does not affect human platelet aggregation or secretion. Thromb Haemost. 2009; 101:1119-1127.
29. Ahn M, Yoder S M, Wang Z, Oh E, Ramalingam L, Tunduguru R, Thurmond D C. The p21-activated kinase (PAK1) is involved in diet-induced beta cell mass expansion and survival in mice and human islets. Diabetologia. 2016; 59:2145-2155.
30. Arqués O, Chicote I, Tenbaum S, Puig I, G. Palmer H. Quantitative Procedure to Analyze Nuclear β-Catenin Using Immunofluorescence Tissue Staining. 2014.
31. Eizirik D L, Mandrup-Poulsen T. A choice of death—the signal-transduction of immune-mediated beta-cell apoptosis. Diabetologia. 2001; 44:2115-2133.
32. Corbett J A, Wang J L, Sweetland M A, Lancaster J R, Jr., McDaniel M L. Interleukin 1 beta induces the formation of nitric oxide by beta-cells purified from rodent islets of Langerhans. Evidence for the beta-cell as a source and site of action of nitric oxide. J Clin Invest. 1992; 90:2384-2391.
33. D'Addio F, Maffi P, Vezzulli P, Vergani A, Mello A, Bassi R, Nano R, Falautano M, Coppi E, Finzi G, D'Angelo A, Fermo I, Pellegatta F, La Rosa S, Magnani G, Piemonti L, Falini A, Folli F, Secchi A, Fiorina P. Islet transplantation stabilizes hemostatic abnormalities and cerebral metabolism in individuals with type 1 diabetes. Diabetes Care. 2014; 37:267-276.
34. Figliolini F, Cantaluppi V, De Lena M, Beltramo S, Romagnoli R, Salizzoni M, Melzi R, Nano R, Piemonti L, Tetta C, Biancone L, Camussi G. Isolation, characterization and potential role in beta cell-endothelium cross-talk of extracellular vesicles released from human pancreatic islets. PLoS One. 2014; 9:e102521.
35. Cianciaruso C, Phelps E A, Pasquier M, Hamelin R, Demurtas D, Alibashe Ahmed M, Piemonti L, Hirosue S, Swartz M A, De Palma M, Hubbell J A, Baekkeskov S. Primary Human and Rat beta-Cells Release the Intracellular Autoantigens GAD65, IA-2, and Proinsulin in Exosomes Together With Cytokine-Induced Enhancers of Immunity. Diabetes. 2017; 66:460-473.
36. Klement G L, Yip T T, Cassiola F, Kikuchi L, Cervi D, Podust V, Italiano J E, Wheatley E, Abou-Slaybi A, Bender E, Almog N, Kieran M W, Folkman J. Platelets actively sequester angiogenesis regulators. Blood. 2009; 113:2835-2842.
37. Zhao Y, Jiang Z, Delgado E, Li H, Zhou H, Hu W, Perez-Basterrechea M, Janostakova A, Tan Q, Wang J, Mao M, Yin Z, Zhang Y, Li Y, Li Q, Zhou J, Li Y, Martinez Revuelta E, Maria Garcia-Gala J, Wang H, Perez-Lopez S, Alvarez-Viejo M, Menendez E, Moss T, Guindi E, Otero J. Platelet-Derived Mitochondria Display Embryonic Stem Cell Markers and Improve Pancreatic Islet beta-cell Function in Humans. Stem Cells Transl Med. 2017; 6:1684-1697.
38. Van Belle T L, Taylor P, von Herrath M G. Mouse Models for Type 1 Diabetes. Drug Discov Today Dis Models. 2009; 6:41-45.
39. Wicker L S, Clark J, Fraser H I, Garner V E, Gonzalez-Munoz A, Healy B, Howlett S, Hunter K, Rainbow D, Rosa R L, Smink L J, Todd J A, Peterson L B. Type 1 diabetes genes and pathways shared by humans and NOD mice. J Autoimmun. 2005; 25 Suppl:29-33.
40. Kabekkodu S P, Bhat S, Radhakrishnan R, Aithal A, Mascarenhas R, Pandey D, Rai L, Kushtagi P, Mundyat G P, Satyamoorthy K. DNA promoter methylation-dependent transcription of the double C2-like domain β (DOC2B) gene regulates tumor growth in human cervical cancer. J Biol Chem. 2014; 289:10637-10649.

1. (canceled)

2. A method of reducing or slowing the development of one or more symptoms associated with T1D, pre-T1D, T2D, or pre-T2D or delaying the onset of one or more symptoms of T1D, pre-T1D, T2D, or pre-T2D in a subject, comprising:
detecting the level of DOC2B expression in a plasma sample collected from the subject,
comparing the level of DOC2B expression with that of a healthy, control subject or with a pre-set threshold level, and
administering one or more T1D or T2D treatments to the subject, if the subject is determined to have a reduced level of DOC2B expression.

3. The method of claim 2, wherein the one or more T1D or T2D treatments include transplanting healthy, functional β cells or pancreatic islets to the subject.

4. A method of assessing early stage pancreatic β-cell destruction or loss of functional β-cells in a subject, comprising:
detecting the level of DOC2B expression in a plasma sample collected from the subject, and
comparing the level of DOC2B expression with that of a healthy, control subject or with a pre-set threshold level, wherein a reduced level of DOC2B expression indicates pancreatic β-cell destruction or loss of functional β-cells in the subject.

5. (canceled)

6. The method of claim 2, wherein detecting the level of DOC2B expression comprises detecting the level of DOC2B protein or the level of DOC2B mRNA in the plasma sample.

7. The method of claim 2, wherein the level of DOC2B protein is determined by ELISA.

8-9. (canceled)

10. The method of claim 2, wherein the level of DOC2B expression is reduced by at least 20%, at least 30%, at least 40%, at least 50%, at least 60%, at least 70%, at least 80%, at least 90%, or at least 95%.

11. The method of claim 2, wherein the subject is human.

12. The method of claim 2, wherein a reduced level of DOC2B expression is detected prior to onset of T1D, pre-T1D, T2D, or pre-T2D in the subject.

13. A method of increasing insulin secretion in a subject with T1D, pre-T1D, T2D, or pre-T2D, comprising:
detecting the level of DOC2B expression in a plasma sample collected from the subject,
comparing the level of DOC2B expression with that of a healthy, control subject or with a pre-set threshold level, and
administering one or more T1D or T2D treatments to the subject, if the subject is determined to have a reduced level of DOC2B expression.

14. The method of claim 13, wherein the one or more T1D or T2D treatments include transplanting healthy, functional β cells or pancreatic islets to the subject.

15. The method of claim 13, wherein detecting the level of DOC2B expression comprises detecting the level of DOC2B protein or the level of DOC2B mRNA in the plasma sample.

16. The method of claim 13, wherein the level of DOC2B protein is determined by ELISA.

17. The method of claim 13, wherein the level of DOC2B expression is reduced by at least 20%, at least 30%, at least 40%, at least 50%, at least 60%, at least 70%, at least 80%, at least 90%, or at least 95%.

18. The method of claim 13, wherein the subject is human.

19. The method of claim 13, wherein a reduced level of DOC2B expression is detected prior to onset of T1D, pre-T1D, T2D, or pre-T2D in the subject.


SEQUENCE LISTING (16 sequences)

SEQ ID NO: 1 - protein, 412 aa, Mus musculus
MTLRRRGEKATISIQEHMAIDVCPGPIRPIKQISDYFPRFPRGLPPTAAPRAPAPPDAPARSPAASASPRSPSDGARDDDEDVDQLFGAYGASPGPSPGPSPARPPAKPPEDEPDVDGYESDDCTALGTLDFSLLYDQENNALHCTISKAKGLKPMDHNGLADPYVKLHLLPGASKANKLRTKTLRNTLNPSWNETLTYYGITDEDMVRKTLRISVCDEDKFRHNEFIGETRVPLKKLKPNHTKTFSICLEKQLPVDKAEDKSLEERGRILISLKYSSQKQGLLVGIVRCAHLAAMDANGYSDPYVKTYLKPDVDKKSKHKTAVKKKTLNPEFNEEFCYEIKHGDLAKKTLEVTVWDYDIGKSNDFIGGVVLGINAKGERLKHWFDCLKNKDKRIERWHTLTNELPGAVLSD

SEQ ID NO: 2 - protein, 412 aa, Rattus norvegicus
MTLRRRGEKATISIQEHMAIDVCPGPIRPIKQISDYFPRFPRGLPPTAAPRASAPPDAPARSPAATAGPRSPSDGARDDDEDVDQLFGAYGASPGPSPGPSPVRPPAKPPEDEPDADGYESDDCTALGTLDFSLLYDQENNALHCTISKAKGLKPMDHNGLADPYVKLHLLPGASKANKLRTKTLRNTLNPSWNETLTYYGITDEDMIRKTLRISVCDEDKFRHNEFIGETRVPLKKLKPNHTKTFSICLEKQLPVDKAEDKSLEERGRILISLKYSSQKQGLLVGIVRCAHLAAMDANGYSDPYVKTYLKPDVDKKSKHKTAVKKKTLNPEFNEEFCYEIKHGDLAKKTLEVTVWDYDIGKSNDFIGGVVLGINAKGERLKHWFDCLKNKDKRIERWHTLTNEIPGAVLSD

SEQ ID NO: 3 - protein, 412 aa, Homo sapiens
MTLRRRGEKATISIQEHMAIDVCPGPIRPIKQISDYFPRFPRGLPPDAGPRAAAPPDAPARPAVAGAGRRSPSDGAREDDEDVDQLFGAYGSSPGPSPGPSPARPPAKPPEDEPDADGYESDDCTALGTLDFSLLYDQENNALHCTITKAKGLKPMDHNGLADPYVKLHLLPGASKANKLRTKTLRNTLNPTWNETLTYYGITDEDMIRKTLRISVCDEDKFRHNEFIGETRVPLKKLKPNHTKTFSICLEKQLPVDKTEDKSLEERGRILISLKYSSQKQGLLVGIVRCAHLAAMDANGYSDPYVKTYLRPDVDKKSKHKTAVKKKTLNPEFNEEFCYEIKHGDLAKKSLEVTVWDYDIGKSNDFIGGVVLGIHAKGERLKHWFDCLKNKDKRIERWHTLTSELPGAVLSD

SEQ ID NO: 4 - protein, 405 aa, Mus musculus
MRGRRGDRMTINIQEHMAINVCPGPIRPIRQISDYFPRRGPGPEGGGGGGGTGCGEAPAHLAPLALAPPAALLGATTPDDGAEVDSYDSDDTTALGTLEFDLLYDQASCMLHCRILRAKGLKPMDFNGLADPYVKLHLLPGACKANKLKTKTQRNTLNPVWNEELTYSGITDDDITHKVLRISVCDEDKLSHNEFIGEIRVPLRRLKPSQKKHFNICLERQVPLPSPSSMSAALRGISCYLKELEQAEQGPGLLEERGRILLSLSYSSRRHGLLVGIVRCAHLAAMDVNGYSDPYVKTYLRPDVDKKSKHKTCVKKKTLNPEFNEEFFYEIELSTLATKTLEVTVWDYDIGKSNDFIGGVSLGPGARGEAQKHWNDCLHQPDTALERWHTLTSELPPAAGAYPLA

SEQ ID NO: 5 - protein, 403 aa, Rattus norvegicus
MRGRRGDRMTINIQEHMAINVCPGPIRPIRQISDYFPRRGPGPEGGGGGRGFGEAPAHLAPLALAPPAALLGATTPDDGAEVDSYDSDDTTALGTLEFDLLYDQASCMLHCRILRAKGLKPMDFNGLADPYVKLHLLPGACKANKLKTKTQRNTLNPVWNEELTYSGITDDDITHKVLRISVCDEDKLSHNEFIGEIRVPLRRLKPSQKKHFNICLERQVPLPSPSSMSAALRGISCYLKELEQAEQGPGLLEERGRILLSLSYSSRRHGLLVGIVRCAHLAAMDVNGYSDPYVKTYLRPDVDKKSKHKTCVKKKTLNPEFNEEFFYEMELSTLATKTLEVTVWDYDIGKSNDFIGGVSLGPGARGEAQKHWRDCLHQPDTAVERWHTLTSELPPAAGALPLA

SEQ ID NO: 6 - protein, 400 aa, Homo sapiens
MRGRRGDRMTINIQEHMAINVCPGPIRPIRQISDYFPRGPGPEGGGGGGGEAPAHLVPLALAPPAALLGATTPEDGAEVDSYDSDDATALGTLEFDLLYDRASCTLHCSILRAKGLKPMDFNGLADPYVKLHLLPGACKANKLKTKTQRNTLNPVWNEDLTYSGITDDDITHKVLRIAVCDEDKLSHNEFIGEIRVPLRRLKPSQKKHFNICLERQVPLASPSSMSAALRGISCYLKELEQAEQGQGLLEERGRILLSLSYSSRRRGLLVGILRCAHLAAMDVNGYSDPYVKTYLRPDVDKKSKHKTCVKKKTLNPEFNEEFFYEIELSTLATKTLEVTVWDYDIGKSNDFIGGVSLGPGARGEARKHWSDCLQQPDAALERWHTLTSELPPAAGALSSA

SEQ ID NO: 7 - protein, 379 aa, synthetic construct
  REGION 1..379: note = Consensus
  VARIANT 7: note = X=E or D
  VARIANT 20: note = X=D or N
  VARIANT 55: note = X=D or E
  VARIANT 78: note = X=D or E
  VARIANT 82: note = X=D or E
  VARIANT 87: note = X=D or E
  VARIANT 98: note = X=D or E
  VARIANT 172: note = X=D or E
  VARIANT 357: note = X=N or Q
MRRRRGXRATINIQEHMAIXVCPGPIRPIRQISDYFPRFGRGLEGGAGGRAAPGXAPARLAPAAAAPRAALDGARTPXDEAXVDGYXSDDCTALGTLXFDLLYDQANNALHCTILRAKGLKPMDHNGLADPYVKLHLLPGACKANKLRTKTQRNTLNPVWNETLTYSGITDXDITRKTLRISVCDEDKLRHNEFIGEIRVPLRRLKPNQKKHFNICLERQLPLDKPEDKSLEERGRILISLKYSSRRQGLLVGIVRCAHLAAMDANGYSDPYVKTYLRPDVDKKSKHKTAVKKKTLNPEFHEEFCYEIEHGDLAKKTLEVTVWDYDIGKSNDFIGGVSLGIGARGEALKHWFDCLKXKDKAIERWHTLTSELPGAAGAD

SEQ ID NO: 8 - protein, 408 aa, synthetic construct
  REGION 1..408: note = Consensus
MTLRRRGEKATISIQEHMAIDVCPGPIRPIKQISDYFPRFPRGLPPTAAPRAAPPDAPARSPAAAGPRSPSDGARDDEDVDQLFGAYGASPGPSPGPSPARPPAKPPEDEPDADGYESDDCTALGTLDFSLLYDQENNALHCTISKAKGLKPMDHNGLADPYVKLHLLPGASKANKLRTKTLRNTLNPSWNETLTYYGITDEDMRKTLRISVCDEDKFRHNEFIGETRVPLKKLKPNHTKTFSICLEKQLPVDKAEDKSLEERGRILISLKYSSQKQGLLVGIVRCAHLAAMDANGYSDPYVKTYLKPDVDKKSKHKTAVKKKTLNPEFNEEFCYEIKHGDLAKKTLEVTVWDYDIGKSNDFIGGVVLGINAKGERLKHWFDCLKNKDKRIERWHTLTNELPGAVLSD

SEQ ID NO: 9 - protein, 22 aa, Homo sapiens
  SITE 22: note = MISC_FEATURE - Cys
DDEDVDQLFGAYGSSPGPSPGX

SEQ ID NO: 10 - protein, 22 aa, Homo sapiens
  SITE 1: note = MISC_FEATURE - Cys
XPSPGPSPARPPAKPPEDEPDA

SEQ ID NO: 11 - protein, 19 aa, Homo sapiens
CLEKQLPVDKTEDKSLEER

SEQ ID NO: 12 - protein, 40 aa, Homo sapiens
CPGPIRPIKQISDYFPRFPRGLPPDAGPRAAAPPDAPARP

SEQ ID NO: 13 - protein, 39 aa, Homo sapiens
  SITE 39: note = MISC_FEATURE - Cys
PPDAPARPAVAGAGRRSPSDGAREDDEDVDQLFGAYGSX

SEQ ID NO: 14 - protein, 36 aa, Homo sapiens
  SITE 1: note = MISC_FEATURE - Cys
XDVDQLFGAYGSSPGPSPGPSPARPPAKPPEDEPDA

SEQ ID NO: 15 - DNA, 20 bp, synthetic construct
  misc_feature 1..20: note = forward primer
  source 1..20: mol_type = other DNA
ccagtaaggcaaataagctc

SEQ ID NO: 16 - DNA, 17 bp, synthetic construct
  misc_feature 1..17: note = reverse primer
  source 1..17: mol_type = other DNA
gggtttcagcttcttca